(19) United States
(12) Patent Application Publication
(10) Pub. No.: US 2009/0035772 A1
(43) Pub. Date: Feb. 5, 2009

(54) Genetic Markers Associated With Scoliosis And Uses Thereof

(75) Inventors: Lesa M. Nelson, Park City, UT (US); Kenneth Ward, Salt Lake City, UT (US)

Correspondence Address: Michael R. Schramm;Axial Biotech, Inc., 2749 East Parleys Way #200, Salt Lake City, UT, 84109, US

(73) Assignee: Axial Biotech, Inc., Salt Lake City, UT (US)

(21) Appl. No.: 12/024,495

(22) Filed: Feb. 1, 2008

Related U.S. Application Data
(63) Continuation-in-part of application No. 11/968,046, filed on Dec. 31, 2007.
Application No. 11/968,046 is a continuation-in-part of application No. 11/968,074, filed on Dec. 31, 2007.

(30) Foreign Application Priority Data
Jul. 3, 2007 (US) PCT/US07/72785
Sep. 11, 2007 (US) PCT/US07/78127
Oct. 19, 2007 (US) PCT/US07/81953

(51) Int. Cl. C12Q 1/68; C07H 21/00; C40B 60/10; A61M 1/00

(52) U.S. Cl. 435/6; 536/24.31; 506/38; 604/540

(57) ABSTRACT

The present invention relates to novel genetic markers associated with scoliosis, risk of developing scoliosis and risk of scoliosis curve progression, and methods and materials for determining whether a human subject has scoliosis, is at risk of developing scoliosis or is at risk of scoliosis curve progression.

### CROSS REFERENCE TO RELATED APPLICATION

This nonprovisional utility application is a continuation-in-part of and claims the benefit under 35 U.S.C. § 120 to co-pending U.S. application Ser. Nos. 11/968,046 and 11/968,074, both filed Dec. 31, 2007 and both of which claim the benefit under 35 U.S.C. § 365(c) of International Patent Application No. PCT/US07/72785 with an international filing date of Jul. 3, 2007 which claims the benefit under 35 U.S.C. § 119(e) of U.S. Provisional Application No. 60/806,498, filed Jul. 3, 2006, and of U.S. Provisional Patent Application No. 60/825,260, filed Sep. 11, 2006, and the benefit under 35 U.S.C. § 365(c) of International Patent Application No. PCT/US07/78127 with an international filing date of Sep. 11, 2007 which claims the benefit under 35 U.S.C. § 119(e) of U.S. Provisional Application No. 60/825,249, filed Sep. 11, 2006, and the benefit under 35 U.S.C. § 365(c) of International Patent Application No. PCT/US07/81953 with an international filing date of Oct. 19, 2007 which claims the benefit under 35 U.S.C. § 119(e) of U.S. Provisional Application No. 60/862,276, filed Oct. 20, 2006, all of which are incorporated, in their entirety, by this reference.

### FIELD OF THE INVENTION

The present invention relates to scoliosis diagnosis and therapy. In particular, the present invention relates to specific single nucleotide polymorphisms (SNPs) in the human genome, and their association with scoliosis and related pathologies.

### BACKGROUND OF THE INVENTION

Scoliosis in one instance refers to adolescent idiopathic scoliosis. In another instance scoliosis refers to either congenital, juvenile, syndromic or any other scoliosis condition. For the purpose of this invention the term scoliosis is used to describe any of these conditions.

Idiopathic scoliosis is the most common pediatric spinal deformity, affecting 2-3% of any human population (5 times the number of females as males), and adolescent idiopathic scoliosis (“AIS”) accounts for approximately 80% of these cases. The current diagnosis for AIS is a clinical finding based on post-symptomatic observation. Current diagnostic regimens, however, are highly inaccurate, inefficient and uncertain, resulting in intense anxiety for patients. The diagnosis of AIS often begins with school screening, which may be sensitive but is never very specific. A study published in the 1980s of 1.5 million Minnesota school children indicated that while nearly 4% of all screened children were referred to an orthopedic surgeon for evaluation, only 1% of the 1.5 million children studied actually had scoliosis (defined as a curvature or Cobb angle >100). Of those 4%, only 1% were actually diagnosed with scoliosis and only 10% of those children progressed to a curve requiring treatment of any kind. Current treatments are only available to patients with a curvature between 25° and 40°. Ten percent of those cases will progress to become candidates for fusion surgery (patients with a curvature >40°). The problem with this paradigm is that there is no reliable method to predict whether an individual's curve will progress and how severe the progression will be.

The current clinical standard of care mandates that all individuals with a clinical diagnosis be followed and potentially treated until they reach skeletal maturity. This inefficiency results in more than 600,000 domestic physician office visits per year for evaluation and observation of scoliosis. Generally, this means visits to an orthopedic surgeon every six months for up to 10 years, with as many as 40 spine X-rays during that period. While it is not unusual for a patient to endure this uncertainty and expense and never actually require treatment, for those who do progress to a curvature >25°, the current option is far from ideal.

Today, the only treatment for patients with a moderate curvature (<40° but >25°) is external bracing. Bracing never corrects a curve, but simply stabilizes the curve during the time an adolescent is growing. Even with perfect compliance, however, the effectiveness of bracing is questionable. Approximately 30% of AIS curves in the 20-29° range will not progress if left untreated and approximately 20% of those who wear their brace compliantly will have curve progression and will require fusion. Thus, 50% of those wearing a brace do not benefit.

Scoliosis is a genetically inherited disease. Genetic variation in DNA sequences is often associated with heritable phenotypes, such as an individual's propensity towards complex disorders. Single nucleotide polymorphisms are the most common form of genetic sequence variations. Detection and analysis of specific genetic mutations, such as single nucleotide polymorphisms (SNPs), which are associated with scoliosis risk, may therefore be used to determine risk of scoliosis, the presence of scoliosis or the progression of scoliosis. Genetic markers that are prognostic for scoliosis can be genotyped early in life and could predict individual response to various risk factors and treatment. Genetic predisposition revealed by genetic analysis of susceptibility genes can provide an integrated assessment of the interaction between genotypes and environmental factors, resulting in synergistically increased prognostic value of diagnostic tests. Thus, pre-symptomatic and early symptomatic genetic testing is expected to be the cornerstone of the paradigmatic shift from late to early stage surgical intervention in spine care using newer minimally invasive devices. A predictive test will provide the information needed to confidently utilize the latest minimally invasive motion preserving technologies in the widest range of patients at the earliest appropriate stage.

Thus, there is an urgent need for novel genetic markers that are predictive of scoliosis and scoliosis progression, particularly in treatment decisions for individuals who are recognized as having a scoliosis. Such genetic markers may enable prognosis of scoliosis in much larger populations compared with the populations which can currently be evaluated by using existing risk factors and biomarkers. The availability of a genetic test may allow, for example, early diagnosis and prognosis of scoliosis, as well as early clinical intervention to mitigate progression of the disease. The use of these genetic markers will also allow selection of subjects for clinical trials involving less invasive treatment methods. The discovery of genetic markers associated with scoliosis will further provide novel targets for therapeutic intervention or preventive treatments of scoliosis and enable the development of new therapeutic agents for treating scoliosis.

### SUMMARY OF THE INVENTION

The present invention relates to the identification of novel polymorphisms, unique combinations of such polymorphisms, and haplotypes of polymorphisms that are associated with scoliosis and related pathologies. The polymorphisms disclosed herein are directly useful as targets for the design of diagnostic reagents and the development of therapeutic agents for use in the diagnosis and treatment of scoliosis and related pathologies.

Based on the identification of particular single nucleotide polymorphisms (SNPS) associated with scoliosis, the present invention also provides methods of detecting these variants as well as the design and preparation of detection reagents needed to accomplish this task. The invention specifically provides novel SNPs in genetic sequences involved in scoliosis, methods of detecting these SNPs in a test sample, methods of identifying individuals who have an altered risk of developing scoliosis or for developing a progressive scoliosis curve based on the presence of a SNP(s) disclosed herein or its encoded product and methods of identifying individuals who are more or less likely to respond to a treatment.

In one embodiment, the present invention provides a method for determining whether a human subject has scoliosis, is at risk of developing scoliosis or is at risk of scoliosis curve progression, comprising: detecting in the genetic material of said subject the presence or absence of one or more protective or high-risk polymorphism selected from the group consisting of the polymorphisms of Table 1 or a polymorphism that is in linkage disequilibrium with a polymorphism of Table 1, wherein the polymorphism is correlated with scoliosis, altered risk of developing scoliosis or altered risk of scoliosis curve progression.

In one embodiment of the invention, the present invention provides polymorphisms having significant allelic association with scoliosis, as set forth in Table 1 or polymorphisms that are in linkage disequilibrium with a polymorphism of Table 1.

In another embodiment, the polymorphisms that are in linkage disequilibrium with a polymorphism of Table 1 are disclosed in Tables 2-203.

In yet another embodiment, the polymorphisms are selected from the polymorphisms of Table 1.

Table 1 provides information identifying the SNPs of the present invention, including SNP “rs” identification numbers (a reference SNP or RefSNP accession ID number), Chi square values, P values, chromosome number, cytogenic band number, base position number of the SNP, sense (+) or antisense (−) strand designation, and genomic-based context sequences that contain SNPs of the present invention.

In a specific embodiment of the present invention, naturally-occurring SNPs in the human genome are provided that are associated with scoliosis. Such SNPs can have a variety of uses in the diagnosis and/or treatment of scoliosis. One aspect of the present invention relates to an isolated nucleic acid molecule comprising a nucleotide sequence in which at least one nucleotide is a SNP disclosed in Tables 1-203. In an alternative embodiment, a nucleic acid of the invention is an amplified polynucleotide, which is produced by amplification of a SNP-containing nucleic acid template.

In yet another embodiment of the invention, a reagent for detecting a SNP in the context of its naturally-occurring flanking nucleotide sequences (which can be, e.g., either DNA or mRNA) is provided. In particular, such a reagent may be in the form of, for example, a hybridization probe or an amplification primer that is useful in the specific detection of a SNP of interest.

Also provided in the invention are kits comprising SNP detection reagents and methods for detecting the SNPs disclosed herein by employing detection reagents. In a specific embodiment, the present invention provides for a method of identifying an individual having an increased or decreased risk of developing scoliosis by detecting the presence or absence of a SNP allele disclosed herein. In another embodiment, a method for diagnosis of scoliosis by detecting the presence or absence of a SNP allele disclosed herein is provided. In yet another embodiment a method for predicting curve progression by detecting the presence or absence of a SNP allele disclosed herein is provided.

In yet another embodiment, the invention also provides a kit comprising SNP detection reagents, and methods for detecting the SNPs disclosed herein by employing detection reagents and a questionnaire of non-genetic clinical factors. In one embodiment, the questionnaire would be completed by a medical professional and gives values for Cobb angle, Risser sign, gender and age. In yet another embodiment, the questionnaire would include any other non-genetic clinical factors known to be associated with the risk of developing scoliosis or the risk for a progressive curve in scoliosis.

Many other uses and advantages of the present invention will be apparent to those skilled in the art upon review of the detailed description of the preferred embodiments herein. Solely for clarity of discussion, the invention is described in the sections below by way of non-limiting examples.

### DETAILED DESCRIPTION OF THE INVENTION

### Definitions

“Haplotype” means a combination of genotypes on the same chromosome occurring in a linkage disequilibrium block. Haplotypes serve as markers for linkage disequilibrium blocks, and at the same time provide information about the arrangement of genotypes within the blocks. Typing of only certain SNPs which serve as tags can, therefore, reveal all genotypes for SNPs located within a block. Thus, the use of haplotypes as tags greatly facilitates identification of candidate genes associated with diseases and drug sensitivity.

“Linkage disequilibrium” or “LD” means that a particular combination of alleles (alternative nucleotides) or genetic markers at two or more different SNP sites are non-randomly co-inherited (i.e., the combination of alleles at the different SNP sites occurs more or less frequently in a population than the separate frequencies of occurrence of each allele or the frequency of a random formation of haplotypes from alleles in a given population). The term “LD” differs from “linkage,” which describes the association of two or more loci on a chromosome with limited recombination between them. LD is also used to refer to any non-random genetic association between allele(s) at two or more different SNP sites. Therefore, when a SNP is in LD with other SNPs, the particular allele of the first SNP often predicts which alleles will be present in those SNPs in LD. LD is generally, but not exclusively, due to the physical proximity of the two loci along a chromosome. Hence, genotyping one of the SNP sites will give almost the same information as genotyping the other SNP site that is in LD. Linkage disequilibrium is caused by fitness interactions between genes or by such non-adaptive processes as population structure, inbreeding, and stochastic effects.

Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome. In one definition, LD can be described mathematically as SNPs that have a D prime value=1 and a LOD score>2.0 or an r-squared value>0.8.

“Linkage disequilibrium block” means a region of the genome that contains multiple SNPs located in proximity to each other and that are transmitted as a block.

“D prime” or D′ (also referred to as the “linkage disequilibrium measure” or “linkage disequilibrium parameter”) means the deviation of the observed allele frequencies from the expected, and is a statistical measure of how well a biometric system can discriminate between different individuals. The larger the D′ value, the better a biometric system is at discriminating between individuals.

“LOD score” is the “logarithm of the odd” score, which is a statistical estimate of whether two genetic loci are physically near enough to each other (or “linked”) on a particular chromosome that they are likely to be inherited together. A LOD score of three or more is generally considered statistically significant evidence of linkage.

“R-squared” or “r²” (also referred to as “correlation coefficient”) is a statistical measure of the degree to which two markers are related. The nearer to 1.0 the r² value is, the more closely the markers are related to each other. R² cannot exceed 1.0. D prime and LOD scores generally follow the above definition for SNPs in LD. R², however, displays a more complex pattern and can vary between about 0.0003 and 1.0 in SNPs that are in LD. (International HapMap Consortium, Nature Oct. 27, 2005; 437:1299-1320).

“Cobb angle” refers to a measure of the curvature of the spine, determined from measurements made on X-ray photographs. Specifically, scoliosis is defined by the Cobb angle. A lateral and rotational spinal curvature of the spine with a Cobb angle of >10° is defined as scoliosis.

“Risser sign” refers to a measurement of skeletal maturity. A Risser sign is defined by the amount of calcification present in the iliac apophysis, divided into quartiles, and measures the progressive ossification from anterolaterally to posteromedially. A Risser grade of 1 signifies up to 25 percent ossification of the iliac apophysis, proceeding to grade 4, which signifies 100 percent ossification (FIG. l). A Risser grade of 5 means the iliac apophysis has fused to the iliac crest after 100 percent ossification. Children usually progress from a Risser grade 1 to a grade 5 over a two-year period during the most rapid skeletal growth.

The present invention provides SNPs associated with scoliosis, nucleic acid molecules containing SNPs, methods and reagents for the detection of the SNPs disclosed herein, uses of these SNPs for the development of detection reagents, and assays or kits that utilize such reagents. The SNPs disclosed herein are useful for diagnosing, screening for, and evaluating predisposition to scoliosis and progression of a scoliosis curve. Additionally, such SNPs are useful in the determining individual subject treatment plans and design of clinical trials of devices for possible use in the treatment of scoliosis. Furthermore, such SNPs and their encoded products are useful targets for the development of therapeutic agents. Furthermore, such SNPs combined with other non-genetic clinical factors such as Cobb angle, Risser sign, age and gender are useful for diagnosing, screening, evaluating predisposition to scoliosis, assessing risk of progression of a scoliosis curve, determining individual subject treatment plans and design of clinical trials of devices for possible use in the treatment of scoliosis.

SNPs

As used herein, the term SNP refers to single nucleotide polymorphisms in DNA. SNPs are usually preceded and followed by highly conserved sequences that vary in less than 1/100 or 1/1000 members of the population. An individual may be homozygous or heterozygous for an allele at each SNP position. A SNP may, in some instances, be referred to as a “cSNP” to denote that the nucleotide sequence containing the SNP is an amino acid “coding” sequence.

A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion or deletion variant referred to as an “indel.”

A synonymous codon change, or silent mutation SNP (terms such as “SNP”, “polymorphism”, “mutation”, “mutant”, “variation”, and “variant” are used herein interchangeably), is one that does not result in a change of amino acid due to the degeneracy of the genetic code. A substitution that changes a codon coding for one amino acid to a codon coding for a different amino acid (i.e., a non-synonymous codon change) is referred to as a mis-sense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. While SNPs can be bi-, tri-, or tetra-allelic, the vast majority of the SNPs are bi-allelic, and are thus often referred to as “bi-allelic markers”, or “di-allelic markers”.

As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases.

Causative SNPs are those SNPs that produce alterations in gene expression or in the expression, structure, and/or function of a gene product, and therefore are most predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic scoliosis.

Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions. Some SNPs that are not causative SNPs nevertheless are in close association with, and therefore segregate with, a disease-causing sequence. In this situation, the presence of a SNP correlates with the presence of, or predisposition to, or an increased risk in developing the scoliosis. These SNPs, although not causative, are nonetheless also useful for diagnostics, scoliosis predisposition screening, scoliosis progression risk and other uses.

An association study of a SNP and a specific disorder involves determining the presence or frequency of the SNP allele in biological samples from individuals with the disorder of interest, such as scoliosis and comparing the information to that of controls (i.e., individuals who do not have the disorder; controls may be also referred to as “healthy” or “normal” individuals) who are preferably of similar age and race. The appropriate selection of patients and controls is important to the success of SNP association studies. Therefore, a pool of individuals with well-characterized phenotypes is extremely desirable.

A SNP may be screened in tissue samples or any biological sample obtained from an affected individual, and compared to control samples, and selected for its increased (or decreased) occurrence in a specific pathological condition, such as pathologies related to scoliosis. Once a statistically significant association is established between one or more SNP(s) and a pathological condition (or other phenotype) of interest, then the region around the SNP can optionally be thoroughly screened to identify the causative genetic locus/sequence(s) (e.g., causative SNP/mutation, gene, regulatory region, etc.) that influences the pathological condition or phenotype. Association studies may be conducted within the general population and are not limited to studies performed on related individuals in affected families (linkage studies).

For diagnostic and prognostic purposes, if a particular SNP site is found to be useful for diagnosing a disease, such as scoliosis, other SNP sites which are in LD with this SNP site would also be expected to be useful for diagnosing the condition. Linkage disequilibrium is described in the human genome as blocks of SNPs along a chromosome segment that do not segregate independently (i.e., that are non-randomly co-inherited). The starting (5′ end) and ending (3′ end) of these blocks can vary depending on the criteria used for linkage disequilibrium in a given database, such as the value of D′ or r² used to determine linkage disequilibrium.

By way of example, Table 1 lists 202 SNPs associated with scoliosis. Furthermore, the SNPs that are in the same linkage disequilibrium block as one of the 202 SNPs in Table 1 may also be useful, either individually, in combination with one of the 202 SNPs in Table 1 or in a haplotype involving one of the 202 SNPs in Table 1. Linkage disequilibrium blocks can be identified in a number of ways such as the SNPbrowser software (v3.5, Applera, Inc., Foster City, Calif.). SNPbrowser is a linkage disequilibrium-guided tool for selection of SNPs. The linkage disequilibrium blocks in SNPbrowser are based on the International HapMap Consortium data and D′ values of linkage disequilibrium.

In accordance with the present invention, SNPs have been identified in a study using a whole-genome case-control approach to identify single nucleotide polymorphisms that were closely associated with the development of idiopathic adolescent scoliosis and specifically progression or non-progression risk to a surgical curve. Table 1 identifies 202 SNPs associated with scoliosis. In addition, SNPs found to be in linkage disequilibrium with (i.e., within the same linkage disequilibrium block as) the scoliosis-associated SNPs of Table 1 can provide haplotypes (i.e., groups of SNPs that are co-inherited) to be readily inferred. The present invention encompasses SNP haplotypes (combinations of SNPs), as well as individual SNPs.

Thus, the present invention provides individual SNPs associated with scoliosis, as well as combinations of SNPs and haplotypes in genetic regions associated with scoliosis, methods of detecting these polymorphisms in a test sample, methods of determining the risk of an individual of having or developing scoliosis and developing a progressive scoliosis curve.

The present invention also provides SNPs associated with scoliosis, as well as SNPs that were previously known in the art, but were not previously known to be associated with scoliosis. Accordingly, the present invention provides novel compositions and methods based on the SNPs disclosed herein, and also provides novel methods of using the known but previously unassociated SNPs in methods relating to scoliosis (e.g., for diagnosing scoliosis. etc.).

Particular SNP alleles of the present invention can be associated with either an increased risk of having or developing scoliosis, or a decreased risk of having or developing scoliosis, or an increased risk of developing a progressive scoliosis curve, or a decreased risk of developing a progressive scoliosis curve. SNP alleles that are associated with a decreased risk may be referred to as “protective” alleles, and SNP alleles that are associated with an increased risk may be referred to as “susceptibility” alleles, “risk factors”, or “high-risk” alleles. Thus, whereas certain SNPs can be assayed to determine whether an individual possesses a SNP allele that is indicative of an increased risk of having or developing scoliosis or a progressive curve (i.e., a susceptibility allele), other SNPs can be assayed to determine whether an individual possesses a SNP allele that is indicative of a decreased risk of having or developing scoliosis or a progressive curve (i.e., a protective allele). Similarly, particular SNP alleles of the present invention can be associated with either an increased or decreased likelihood of responding to a particular treatment. The term “altered” may be used herein to encompass either of these two possibilities (e.g., an increased or a decreased risk/likelihood).

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the complementary thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers may be designed to hybridize to either strand and SNP genotyping methods disclosed herein may generally target either strand. Throughout the specification, in identifying a SNP position, reference is generally made to the forward or “sense” strand, solely for the purpose of convenience. Since endogenous nucleic acid sequences exist in the form of a double helix (a duplex comprising two complementary nucleic acid strands), it is understood that the SNPs disclosed herein will have counterpart nucleic acid sequences and SNPs associated with the complementary “reverse” or “antisense” nucleic acid strand. Such complementary nucleic acid sequences, and the complementary SNPs present in those sequences, are also included within the scope of the present invention.

The present invention provides methods for utilizing the SNPs disclosed in Tables 1-203 for determining whether a human subject has scoliosis, is at risk of developing scoliosis or is at risk of scoliosis curve progression. In some embodiments, the methods of the invention comprise the step of detecting in the genetic material of said subject the presence or absence of one or more protective or high-risk polymorphism selected from the group consisting of the polymorphisms of Table 1 or a polymorphism that is in linkage disequilibrium with a polymorphism of Table 1, wherein the polymorphism is correlated with scoliosis, altered risk of developing scoliosis or altered risk of scoliosis curve progression. In other embodiments, the polymorphism that is in linkage disequilibrium with a polymorphism of Table 1 is selected from the polymorphisms of Tables 2-203. In other embodiments, the polymorphism is selected from the polymorphisms of Table 1.

In other embodiments, the methods further comprise the step of evaluating the risk associated with one or more non-genetic clinical factors selected from the group consisting of Cobb angle, age, Risser sign, age at menarche, gender and other factors associated with scoliosis.

In other embodiments, the method of detecting in a nucleic acid molecule a polymorphism that is correlated with scoliosis, altered risk of developing scoliosis or altered risk of scoliosis curve progression, comprises contacting a test sample with a polynucleotide sequence that specifically hybridizes under stringent hybridization conditions to a polynucleotide sequence having one or more protective or high-risk polymorphism selected from the group consisting of the polymorphisms of Table 1 or a polymorphism that is in linkage disequilibrium with a polymorphism of Table 1 or a complement thereof, wherein the polymorphism is correlated with scoliosis, altered risk of developing scoliosis or altered risk of scoliosis curve progression, and detecting the formation of a hybridized duplex.

With respect to the above methods, the polymorphism may be correlated with an increased risk of scoliosis curve progression in a human subject with a scoliosis curve.

The above methods may further comprise the step of correlating the polymorphism with an appropriate medical treatment, including the use of medical devices or pharmaceuticals, in a human subject known to have a scoliosis curve or who has been determined to be at risk for scoliosis or scoliosis curve progression.

The above methods may further comprise the step of selecting human subjects for clinical trials involving either medical devices or pharmaceuticals for use in the treatment of scoliosis.

In the above methods, the polymorphism may be correlated with presymptomatic risk of developing scoliosis in a human subject. The human subject may be an adult or may be a human fetus.

In the above methods, the step of assessing scoliosis risk may be by determining whether each of a set of independent variables has a unique predictive relationship to a dichotomous dependent variable. The step of assessing scoliosis risk may, for example, comprise an algorithm comprising a logistic regression analysis.

Amplified Nucleic Acid Molecules

The present invention further provides amplified polynucleotides containing the nucleotide sequence of a polymorphism selected from the polymorphisms of Table 1 or a polymorphism that is in linkage disequilibrium with a polymorphism of Table 1 or a complement thereof, wherein the amplified polynucleotide is greater than about 16 nucleotides in length. The polymorphism may be in linkage disequilibrium with a polymorphism of Table 1 and is selected from the polymorphisms of Tables 2-203. The polymorphism may also be selected from the polymorphisms of Table 1.

Isolated Nucleic Acid Molecules and SNP Detection Reagents & Kits

Tables 1-203 provide information identifying the SNPs of the present invention that are associated with scoliosis. Table 1 includes additional information about the SNP, such as nucleotide substitution, chromosome number, cytogenetic band and p-values from the current invention, as well as the genomic-based SNP context sequences. The context sequences generally include approximately 25 nucleotides upstream (5′) plus 25 nucleotides downstream (3′) of each SNP position, and the alternative nucleotides (alleles) at each SNP position.

Isolated Nucleic Acid Molecules

The present invention further provides isolated polynucleotide molecules that specifically hybridize to a polynucleotide molecule containing the nucleotide sequence of a polymorphism selected from any one of the polymorphisms of Tables 1 or a polymorphism that is in linkage disequilibrium with a polymorphism of Table 1 or a complement thereof. In some embodiments, the polymorphism that is in linkage disequilibrium with a polymorphism of Table 1 is selected from the polymorphisms of Tables 2-203. In other embodiments, the polymorphism is selected from the polymorphisms of Table 1.

In particular embodiments, the isolated polynucleotides of the present invention may be from about 8-70 nucleotides in length.

In some embodiments the polynucleotide is an allele-specific probe. In other embodiments, the polynucleotide is an allele-specific primer.

The present invention provides isolated nucleic acid molecules that contain one or more SNPs disclosed Tables 1-203. Preferred isolated nucleic acid molecules contain one or more SNPs identified in Table 1. Isolated nucleic acid molecules containing one or more SNPs disclosed in Table 1 may be interchangeably referred to throughout the present text as “SNP-containing nucleic acid molecules.” The isolated nucleic acid molecules of the present invention also include probes and primers (which are described in greater detail below in the section entitled “SNP Detection Reagents”), which may be used for assaying the disclosed SNPs, and isolated full-length genes, transcripts, cDNA molecules, and fragments thereof, which may be used for such purposes as expressing an encoded protein.

As used herein, an “isolated nucleic acid molecule” generally is one that contains a SNP of the present invention or one that hybridizes to such molecule such as a nucleic acid with a complementary sequence, and is separated from most other nucleic acids present in the natural source of the nucleic acid molecule. Moreover, an “isolated” nucleic acid molecule, such as a cDNA molecule containing a SNP of the present invention, can be substantially free of other cellular material, or culture medium when produced by recombinant techniques, or chemical precursors or other chemicals when chemically synthesized. A nucleic acid molecule can be fused to other coding or regulatory sequences and still be considered “isolated.” Nucleic acid molecules present in non-human transgenic animals, which do not naturally occur in the animal, are also considered “isolated.” For example, recombinant DNA molecules contained in a vector are considered “isolated.” Further examples of “isolated” DNA molecules include recombinant DNA molecules maintained in heterologous host cells, and purified (partially or substantially) DNA molecules in solution. Isolated RNA molecules include in vivo or in vitro RNA transcripts of the isolated SNP-containing DNA molecules of the present invention. Isolated nucleic acid molecules according to the present invention further include such molecules produced synthetically.

Generally, an isolated SNP-containing nucleic acid molecule comprises one or more SNP positions disclosed by the present invention with flanking nucleotide sequences on either side of the SNP positions. A flanking genomic context sequence can include nucleotide residues that are naturally associated with the SNP site and/or heterologous nucleotide sequences. The flanking sequence may be up to about 100, 60, 50, 30, 25, 20, 15, 10, 8, or 4 nucleotides (or any other length in-between) on either side of a SNP position.

For full-length genes and entire protein-coding sequences, a SNP flanking sequence can be, for example, up to about 5 KB, 4 KB, 3 KB, 2 KB, or 1 KB on either side of the SNP. Furthermore, in such instances, the isolated nucleic acid molecule comprises exonic sequences (including protein-coding and/or non-coding exonic sequences), but may also include intronic sequences. Thus, any protein coding sequence may be either contiguous or separated by introns. The important point is that the nucleic acid is isolated from remote and unimportant flanking sequences and is of appropriate length such that it can be subjected to the specific manipulations or uses described herein such as recombinant protein expression, preparation of probes and primers for assaying the SNP position, and other uses specific to the SNP-containing nucleic acid sequences.

An isolated SNP-containing nucleic acid molecule can comprise, for example, a full-length gene or transcript, such as a gene isolated from genomic DNA (e.g., by cloning or PCR amplification), a cDNA molecule, or an mRNA transcript molecule. Furthermore, fragments of such full-length genes and transcripts that contain one or more SNPs disclosed herein are also encompassed by the present invention, and such fragments may be used, for example, to express any part of a protein, such as a particular functional domain or an antigenic epitope.

Thus, the present invention also encompasses fragments of the nucleic acid sequences provided in Table 1, contiguous nucleotide sequence at least about 8 or more nucleotides, more preferably at least about 12 or more nucleotides, and even more preferably at least about 16 or more nucleotides. Further, a fragment could comprise at least about 18, 20, 22, 25, 30, 40, 50, 60, 100, 250 or 500 (or any other number in-between) nucleotides in length. The length of the fragment will be based on its intended use. For example, the fragment can be useful as a polynucleotide probe or primer. Such fragments can be isolated using the nucleotide sequences provided in Table 1 for the synthesis of a polynucleotide probe. A labeled probe can then be used, for example, to screen a cDNA library, genomic DNA library, or mRNA to isolate nucleic acid corresponding to the coding region. Further, primers can be used in amplification reactions, such as for purposes of assaying one or more SNPs sites or for cloning specific regions of a gene.

An isolated nucleic acid molecule of the present invention further encompasses a SNP-containing polynucleotide that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874, 1990). Based on such methodologies, a person skilled in the art can readily design primers in any suitable regions 5′ and 3′ to a SNP disclosed herein. Such primers may be used to amplify DNA of any length so long that it contains the SNP of interest in its sequence.

As used herein, an “amplified polynucleotide” of the invention is a SNP-containing nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 16 nucleotides in length. More typically, an amplified polynucleotide is at least about 20 nucleotides in length. In a preferred embodiment of the invention, an amplified polynucleotide is at least about 30 nucleotides in length. In a more preferred embodiment of the invention, an amplified polynucleotide is at least about 32, 40, 45, 50, or 60 nucleotides in length. In yet another preferred embodiment of the invention, an amplified polynucleotide is at least about 100, 200, or 300 nucleotides in length. While the total length of an amplified polynucleotide of the invention can be as long as an exon, an intron or the entire gene where the SNP of interest resides, an amplified product is typically no greater than about 1,000 nucleotides in length (although certain amplification methods may generate amplified products greater than 1000 nucleotides in length). More preferably, an amplified polynucleotide is not greater than about 600 nucleotides in length. It is understood that irrespective of the length of an amplified polynucleotide, a SNP of interest may be located anywhere along its sequence.

In a specific embodiment of the invention, the amplified product is at least about 201 nucleotides in length, comprises one of the nucleotide sequences shown in Table 1. Such a product may have additional sequences on its 5′ end or 3′ end or both. In another embodiment, the amplified product is about 101 nucleotides in length, and it contains a SNP disclosed herein. Generally, the SNP is located at the middle of the amplified product (e.g., at position 101 in an amplified product that is 201 nucleotides in length, or at position 51 in an amplified product that is 101 nucleotides in length), or within 1, 2, 3, 4, 5, 6, 7, 8, 9, 10, 12, 15, or 20 nucleotides from the middle of the amplified product (however, as indicated above, the SNP of interest may be located anywhere along the length of the amplified product).

The present invention provides isolated nucleic acid molecules that comprise, consist of, or consist essentially of one or more polynucleotide sequences that contain one or more SNPs disclosed herein, complements thereof, and SNP-containing fragments thereof.

Accordingly, the present invention provides nucleic acid molecules that consist of any of the nucleotide sequences shown in Table 1. A nucleic acid molecule consists of a nucleotide sequence when the nucleotide sequence is the complete nucleotide sequence of the nucleic acid molecule.

The present invention further provides nucleic acid molecules that consist essentially of any of the nucleotide sequences shown in Table 1. A nucleic acid molecule consists essentially of a nucleotide sequence when such a nucleotide sequence is present with only a few additional nucleotide residues in the final nucleic acid molecule.

The present invention further provides nucleic acid molecules that comprise any of the nucleotide sequences shown in Table 1. A nucleic acid molecule comprises a nucleotide sequence when the nucleotide sequence is at least part of the final nucleotide sequence of the nucleic acid molecule. In such a fashion, the nucleic acid molecule can be only the nucleotide sequence or have additional nucleotide residues, such as residues that are naturally associated with it or heterologous nucleotide sequences. Such a nucleic acid molecule can have one to a few additional nucleotides or can comprise many more additional nucleotides. A brief description of how various types of these nucleic acid molecules can be readily made and isolated are well known to those of ordinary skill in the art (Sambrook and Russell, 2000, Molecular Cloning: A Laboratory Manual, Cold Spring Harbor Press, NY).

Isolated nucleic acid molecules can be in the form of RNA, such as mRNA, or in the form DNA, including cDNA and genomic DNA, which may be obtained, for example, by molecular cloning or produced by chemical synthetic techniques or by a combination thereof (Sambrook and Russell, 2000, Molecular Cloning: A Laboratory Manual, Cold Spring Harbor Press, NY). Furthermore, isolated nucleic acid molecules, particularly SNP detection reagents such as probes and primers, can also be partially or completely in the form of one or more types of nucleic acid analogs, such as peptide nucleic acid (PNA) (U.S. Pat. Nos. 5,539,082; 5,527,675; 5,623,049; 5,714,331). The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled, for example, from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well known in the art (see, e.g., Corey, “Peptide nucleic acids: expanding the scope of nucleic acid recognition”, Trends Biotechnol. 1997 June; 15(6):224-9, and Hyrup et al., “Peptide nucleic acids (PNA): synthesis, properties and potential applications”, Bioorg Med Chem. 1996 January; 4(1):5-23).

The present invention encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes) for detecting one or more SNPs identified in Tables 1-203. Furthermore, kits/systems (such as beads, arrays, etc.) that include these analogs are also encompassed by the present invention.

Additional examples of nucleic acid modifications that improve the binding properties and/or stability of a nucleic acid include the use of base analogs such as inosine, intercalators (U.S. Pat. No. 4,835,263) and the minor groove binders (U.S. Pat. No. 5,801,115). Thus, references herein to nucleic acid molecules, SNP-containing nucleic acid molecules, SNP detection reagents (e.g., probes and primers), and oligonucleotides/polynucleotides include PNA oligomers and other nucleic acid analogs. Other examples of nucleic acid analogs and alternative/modified nucleic acid chemistries known in the art are described in Current Protocols in Nucleic Acid Chemistry, John Wiley & Sons, N.Y. (2002).

Further variants of the nucleic acid molecules disclosed in Tables 1-203, such as naturally occurring allelic variants (as well as orthologs and paralogs) and synthetic variants produced by mutagenesis techniques, can be identified and/or produced using methods well known in the art. Such further variants can comprise a nucleotide sequence that shares at least 70-80%, 80-85%, 85-90%, 91%, 92%, 93%, 94%, 95%, 96%, 97%, 98%, or 99% sequence identity with a nucleic acid sequence disclosed in Table 1 (or a fragment thereof) and that includes a novel SNP allele disclosed in Table 1. Thus, the present invention specifically contemplates isolated nucleic acid molecule that have a certain degree of sequence variation compared with the sequences shown in Table 1, but that contain a novel SNP allele disclosed herein. In other words, as long as an isolated nucleic acid molecule contains a novel SNP allele disclosed herein, other portions of the nucleic acid molecule that flank the novel SNP allele can vary to some degree from the specific genomic and context sequences shown in Tables 1-203.

To determine the percent identity of two nucleotide sequences of two molecules that share sequence homology, the sequences are aligned for optimal comparison purposes (e.g., gaps can be introduced in one or both of a first and a second nucleic acid sequence for optimal alignment and non-homologous sequences can be disregarded for comparison purposes). In a preferred embodiment, at least 30%, 40%, 50%, 60%, 70%, 80%, or 90% or more of the length of a reference sequence is aligned for comparison purposes. The nucleotides at corresponding nucleotide positions are then compared. When a position in the first sequence is occupied by the same nucleotide as the corresponding position in the second sequence, then the molecules are identical at that position (as used herein, nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, N. J., 1994; Sequence Analysis in Molecular Biology, von Heinje, G., Academic Press, 1987; and Sequence Analysis Primer, Gribskov, M. and Devereux, J., eds., M Stockton Press, New York, 1991).

In one particular embodiment, the percent identity between two nucleotide sequences is determined using the GAP program in the GCG software package (Devereux, J., et al., Nucleic Acids Res. 12(1):387 (1984)), using an NWSgapdna.CMP matrix and a gap weight of 40, 50, 60, 70, or 80 and a length weight of 1, 2, 3, 4, 5, or 6. In another embodiment, the percent identity between two nucleotide sequences is determined using the algorithm of E. Myers and W. Miller (CABIOS, 4:11-17 (1989)) which has been incorporated into the ALIGN program (version 2.0), using a PAM120 weight residue table, a gap length penalty of 12, and a gap penalty of 4.

The nucleotide sequences of the present invention can further be used as a “query sequence” to perform a search against sequence databases to, for example, identify other family members or related sequences. Such searches can be performed using the NBLAST and XBLAST programs (version 2.0) of Altschul, et al. (J. Mol. Biol. 215:403-10 (1990)). BLAST nucleotide searches can be performed with the NBLAST program, score=100, wordlength=12 to obtain nucleotide sequences homologous to the nucleic acid molecules of the invention. To obtain gapped alignments for comparison purposes, Gapped BLAST can be utilized as described in Altschul et al. (Nucleic Acids Res. 25(17):3389-3402 (1997)). When utilizing BLAST and gapped BLAST programs, the default parameters of the respective programs (e.g., XBLAST and NBLAST) can be used. In addition to BLAST, examples of other search and sequence comparison programs used in the art include, but are not limited to, FASTA (Pearson, Methods Mol. Biol. 25, 365-389 (1994)) and KERR (Dufresne et al., Nat Biotechnol 2002 December; 20(12): 1269-71). For further information regarding bioinformatics techniques, see Current Protocols in Bioinformatics, John Wiley & Sons, Inc., N.Y.

SNP Detection Reagents

In a specific aspect of the present invention, the SNPs disclosed herein can be used for the design of SNP detection reagents. As used herein, a “SNP detection reagent” is a reagent that specifically detects a specific target SNP position disclosed herein, and that is preferably specific for a particular nucleotide (allele) of the target SNP position (i.e., the detection reagent preferably can differentiate between different alternative nucleotides at a target SNP position, thereby allowing the identity of the nucleotide present at the target SNP position to be determined). Typically, such detection reagent hybridizes to a target SNP- containing nucleic acid molecule by complementary base-pairing in a sequence specific manner, and discriminates the target variant sequence from other nucleic acid sequences such as an art-known form in a test sample. An example of a detection reagent is a probe that hybridizes to a target nucleic acid containing one or more of the SNPs disclosed herein. In a preferred embodiment, such a probe can differentiate between nucleic acids having a particular nucleotide (allele) at a target SNP position from other nucleic acids that have a different nucleotide at the same target SNP position. In addition, a detection reagent may hybridize to a specific region 5′ and/or 3′ to a SNP position, particularly a region corresponding to the context sequences provided in the SNPs disclosed herein. Another example of a detection reagent is a primer which acts as an initiation point of nucleotide extension along a complementary strand of a target polynucleotide. The SNP sequence information provided herein is also useful for designing primers, e.g. allele-specific primers, to amplify (e.g., using PCR) any SNP of the present invention.

In one preferred embodiment of the invention, a SNP detection reagent is a synthetic polynucleotide molecule, such as an isolated or synthetic DNA or RNA polynucleotide probe or primer or PNA oligomer, or a combination of DNA, RNA and/or PNA that hybridizes to a segment of a target nucleic acid molecule containing a SNP identified herein. A detection reagent in the form of a polynucleotide may optionally contain modified base analogs, intercalators or minor groove binders. Multiple detection reagents such as probes may be, for example, affixed to a solid support (e.g., arrays or beads) or supplied in solution (e.g., probe/primer sets for enzymatic reactions such as PCR, RT-PCR, TaqMan assays, or primer-extension reactions) to form a SNP detection kit.

A probe or primer typically is a substantially purified oligonucleotide. Such oligonucleotide typically comprises a region of complementary nucleotide sequence that hybridizes under stringent conditions to at least about 8, 10, 12, 16, 18, 20, 22, 25, 30, 40, 50, 60, 100 (or any other number in-between) or more consecutive nucleotides in a target nucleic acid molecule. Depending on the particular assay, the consecutive nucleotides can either include the target SNP position, or be a specific region in close enough proximity 5′ and/or 3′ to the SNP position to carry out the desired assay.

Other preferred primer and probe sequences can readily be determined using the nucleotide sequences disclosed herein. It will be apparent to one of skill in the art that such primers and probes are directly useful as reagents for genotyping the SNPs of the present invention, and can be incorporated into any kit/system format.

In order to produce a probe or primer specific for a target SNP-containing sequence, the gene/transcript and/or context sequence surrounding the SNP of interest is typically examined using a computer algorithm which starts at the 5′ or at the 3′ end of the nucleotide sequence. Typical algorithms will then identify oligomers of defined length that are unique to the gene/SNP context sequence, have a GC content within a range suitable for hybridization, lack predicted secondary structure that may interfere with hybridization, and/or possess other desired characteristics or that lack other undesired characteristics.

A primer or probe of the present invention is typically at least about 8 nucleotides in length. In one embodiment of the invention, a primer or a probe is at least about 10 nucleotides in length. In a preferred embodiment, a primer or a probe is at least about 12 nucleotides in length. In a more preferred embodiment, a primer or probe is at least about 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length (see the section below entitled “SNP Detection Kits and Systems”).

For analyzing SNPs, it may be appropriate to use oligonucleotides specific for alternative SNP alleles. Such oligonucleotides which detect single nucleotide variations in target sequences may be referred to by such terms as “allele-specific oligonucleotides”, “allele-specific probes”, or “allele-specific primers”. The design and use of allele-specific probes for analyzing polymorphisms is described in, e.g., Mutation Detection A Practical Approach, ed. Cotton et al. Oxford University Press, 1998; Saiki et al., Nature 324, 163-166 (1986); Dattagupta, EP235,726; and Saiki, WO 89/11548.

While the design of each allele-specific primer or probe depends on variables such as the precise composition of the nucleotide sequences flanking a SNP position in a target nucleic acid molecule, and the length of the primer or probe, another factor in the use of primers and probes is the stringency of the condition under which the hybridization between the probe or primer and the target sequence is performed. Higher stringency conditions utilize buffers with lower ionic strength and/or a higher reaction temperature, and tend to require a more perfect match between probe/primer and a target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an allele-specific probe are as follows: Prehybridization with a solution containing 5+ standard saline phosphate EDTA (SSPE), 0.5% NaDodSO₄ (SDS) at 55° C., and incubating probe with target nucleic acid molecules in the same solution at the same temperature, followed by washing with a solution containing 2×SSPE, and 0.1% SDS at 55° C. or room temperature.

Moderate stringency hybridization conditions may be used for allele-specific primer extension reactions with a solution containing, e.g., about 50 mM KCl at about 46° C. Alternatively, the reaction may be carried out at an elevated temperature such as 60° C. In another embodiment, a moderately stringent hybridization condition suitable for oligonucleotide ligation assay (OLA) reactions wherein two probes are ligated if they are completely complementary to the target sequence may utilize a solution of about 100 mM KCl at a temperature of 46° C.

In a hybridization-based assay, allele-specific probes can be designed that hybridize to a segment of target DNA from one individual but do not hybridize to the corresponding segment from another individual due to the presence of different polymorphic forms (e.g., alternative SNP alleles/nucleotides) in the respective DNA segments from the two individuals. Hybridization conditions should be sufficiently stringent that there is a significant detectable difference in hybridization intensity between alleles, and preferably an essentially binary response, whereby a probe hybridizes to only one of the alleles or significantly more strongly to one allele. While a probe may be designed to hybridize to a target sequence that contains a SNP site such that the SNP site aligns anywhere along the sequence of the probe, the probe is preferably designed to hybridize to a segment of the target sequence such that the SNP site aligns with a central position of the probe (e.g., a position within the probe that is at least three nucleotides from either end of the probe). This design of probe generally achieves good discrimination in hybridization between different allelic forms.

In another embodiment, a probe or primer may be designed to hybridize to a segment of target DNA such that the SNP aligns with either the 5′ most end or the 3′ most end of the probe or primer. In a specific preferred embodiment which is particularly suitable for use in an oligonucleotide ligation assay (U.S. Pat. No. 4,988,617), the most 3′nucleotide of the probe aligns with the SNP position in the target sequence.

Oligonucleotide probes and primers may be prepared by methods well known in the art. Chemical synthetic methods include, but are limited to, the phosphotriester method described by Narang et al., 1979, Methods in Enzymology 68:90; the phosphodiester method described by Brown et al., 1979, Methods in Enzymology 68:109, the diethylphosphoamidate method described by Beaucage et al., 1981, Tetrahedron Letters 22:1859; and the solid support method described in U.S. Pat. No. 4,458,066.

Allele-specific probes are often used in pairs (or, less commonly, in sets of 3 or 4, such as if a SNP position is known to have 3 or 4 alleles, respectively, or to assay both strands of a nucleic acid molecule for a target SNP allele), and such pairs may be identical except for a one nucleotide mismatch that represents the allelic variants at the SNP position. Commonly, one member of a pair perfectly matches a reference form of a target sequence that has a more common SNP allele (i.e., the allele that is more frequent in the target population) and the other member of the pair perfectly matches a form of the target sequence that has a less common SNP allele (i.e., the allele that is rarer in the target population). In the case of an array, multiple pairs of probes can be immobilized on the same support for simultaneous analysis of multiple different polymorphisms.

In one type of PCR-based assay, an allele-specific primer hybridizes to a region on a target nucleic acid molecule that overlaps a SNP position and only primes amplification of an allelic form to which the primer exhibits perfect complementarity (Gibbs, 1989, Nucleic Acid Res. 17 2427-2448). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer (see, e.g., WO 93/22456). This PCR-based assay can be utilized as part of the TaqMan assay, described below.

In a specific embodiment of the invention, a primer of the invention contains a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In a preferred embodiment, the mismatched nucleotide in the primer is the second from the last nucleotide at the 3′-most position of the primer. In a more preferred embodiment, the mismatched nucleotide in the primer is the last nucleotide at the 3′-most position of the primer.

In another embodiment of the invention, a SNP detection reagent of the invention is labeled with a fluorogenic reporter dye that emits a detectable signal. While the preferred reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red. [00103] In yet another embodiment of the invention, the detection reagent may be further labeled with a quencher dye such as Tamra, especially when the reagent is used as a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl. 4:357-362; Tyagi et al., 1996, Nature Biotechnology 14: 303-308; Nazarenko et al., 1997, Nucl. Acids Res. 25:2516-2521; U.S. Pat. Nos. 5,866,336 and 6,117,635).

The detection reagents of the invention may also contain other labels, including but not limited to, biotin for streptavidin binding and oligonucleotide for binding to another complementary oligonucleotide such as pairs of zipcodes.

The present invention also contemplates reagents that do not contain (or that are complementary to) a SNP nucleotide identified herein but that are used to assay one or more SNPs disclosed herein. For example, primers that flank, but do not hybridize directly to a target SNP position provided herein are useful in primer extension reactions in which the primers hybridize to a region adjacent to the target SNP position (i.e., within one or more nucleotides from the target SNP site). During the primer extension reaction, a primer is typically not able to extend past a target SNP site if a particular nucleotide (allele) is present at that target SNP site, and the primer extension product can readily be detected in order to determine which SNP allele is present at the target SNP site. For example, particular ddNTPs are typically used in the primer extension reaction to terminate primer extension once a ddNTP is incorporated into the extension product (a primer extension product which includes a ddNTP at the 3′-most end of the primer extension product, and in which the ddNTP corresponds to a SNP disclosed herein, is a composition that is encompassed by the present invention). Thus, reagents that bind to a nucleic acid molecule in a region adjacent to a SNP site, even though the bound sequences do not necessarily include the SNP site itself, are also encompassed by the present invention.

SNP Detection Kits and Systems

A person skilled in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art.

The kits of the present invention may be used for detecting a nucleic acid polymorphism indicative of an altered risk in a symptomatic or presymptomatic scoliosis subject. Such kits may comprise a polynucleotide having a SNP of Table 1, a SNP that is in linkage disequilibrium with a SNP of Table 1 or a SNP of Tables 2-203, enzymes, buffers, and reagents used to detect genetic polymorphisms. The kits may further comprise a questionnaire of non-genetic clinical factors.

The terms “kits” and “systems”, as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). Accordingly, the present invention further provides SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more SNPs of the present invention. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment of the present invention, kits are provided which contain the necessary reagents to carry out one or more assays to detect one or more SNPs disclosed herein. In a preferred embodiment of the present invention, SNP detection kits/systems are in the form of nucleic acid arrays, or compartmentalized kits, including microfluidic/lab-on-a-chip systems.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is a SNP of the present invention. In some kits/systems, the allele-specific probes are immobilized to a substrate such as an array or bead. For example, the same substrate can comprise allele-specific probes for detecting at least 1; 10; 100; 1000; 10,000; 100,000; 500,000 (or any other number inbetween) or substantially all of the SNPs disclosed herein.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832, Chee et al., PCT application W095/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522.

Nucleic acid arrays are reviewed in the following references: Zammatteo et al., “New chips for molecular biology and diagnostics”, Biotechnol Annu Rev. 2002;8:85-101; Sosnowski et al., “Active microelectronic array system for DNA hybridization, genotyping and pharmacogenomic applications”, Psychiatr Genet. 2002 December; 12(4): 181-92; Heller, “DNA microarray technology: devices, systems, and applications”, Annu Rev Biomed Eng. 2002;4:129-53. Epub 2002 Mar. 22; Kolchinsky et al., “Analysis of SNPs and other genomic variations using gel-based chips”, Hum Mutat. 2002 April; 19(4):343-60; and McGall et al., “High-density genechip oligonucleotide probe arrays”, Adv Biochem Eng Biotechnol. 2002;77:21-42.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length. The microarray or detection kit can contain polynucleotides that cover the known 5′or 3′ sequence of the target SNP site, sequential polynucleotides that cover the full-length sequence of a gene/transcript; or unique polynucleotides selected from particular areas along the length of a target gene/transcript sequence, particularly areas corresponding to one or more SNPs disclosed herein. Polynucleotides used in the microarray or detection kit can be specific to a SNP or SNPs of interest (e.g., specific to a particular SNP allele at a target SNP site, or specific to particular SNP alleles at multiple different SNP sites), or specific to a polymorphic gene/transcript or genes/transcripts of interest.

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions may be preferable when using, for example, nucleic acid arrays of allele-specific probes for SNP detection. Such high stringency conditions are described in the preceding section, and are well known to those skilled in the art and can be found in, for example, Current Protocols in Molecular Biology, John Wiley & Sons, N.Y. (1989), 6.3.1-6.3.6.

In other embodiments, the arrays are used in conjunction with chemiluminescent detection technology. The following patents and patent applications, which are all hereby incorporated by reference, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5994073, 5981768, 5871938, 5843681, 5800999, and 5773628 describe methods and compositions of dioxetane for performing chemiluminescent detection; and U.S. published application US2002/0110828 discloses methods and compositions for microarray controls.

In one embodiment of the invention, a nucleic acid array can comprise an array of probes of about 15-25 nucleotides in length. In further embodiments, a nucleic acid array can comprise any number of probes, in which at least one probe is capable of detecting one or more SNPs disclosed in Tables 1-203 and/or at least one probe comprises a fragment of one of the sequences selected from the group consisting of those disclosed herein, and sequences complementary thereto, said fragment comprising at least about 8 consecutive nucleotides, preferably 10, 12, 15, 16, 18, 20, more preferably 22, 25, 30, 40, 47, 50, 55, 60, 65, 70, 80, 90, 100, or more consecutive nucleotides (or any other number in-between) and containing (or being complementary to) a SNP. In some embodiments, the nucleotide complementary to the SNP site is within 5, 4, 3, 2, or 1 nucleotide from the center of the probe, more preferably at the center of said probe.

A polynucleotide probe can be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other number which lends itself to the efficient use of commercially available instrumentation.

Using such arrays or other kits/systems, the present invention provides methods of identifying the SNPs disclosed herein in a test sample. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

A SNP detection kit/system of the present invention may include components that are used to prepare nucleic acids from a test sample for the subsequent amplification and/or detection of a SNP-containing nucleic acid molecule. Such sample preparation components can be used to produce nucleic acid extracts, including DNA and/or RNA, extracts from any bodily fluids. In a preferred embodiment of the invention, the bodily fluid is blood, saliva or buccal swabs. The test samples used in the above-described methods will vary based on such factors as the assay format, nature of the detection method, and the specific tissues, cells or extracts used as the test sample to be assayed. Methods of preparing nucleic acids are well known in the art and can be readily adapted to obtain a sample that is compatible with the system utilized.

In yet another form of the kit in addition to reagents for preparation of nucleic acids and reagents for detection of one of the SNPs of this invention, the kit may include a questionnaire inquiring about non-genetic clinical factors such as Cobb angle, Risser sign, age, gender or any other non-genetic clinical factors known to be associated with scoliosis.

Another form of kit contemplated by the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting one or more SNPs of the present invention, one or more containers which contain wash reagents (such as phosphate buffered saline, Tris-buffers, etc.), and one or more containers which contain the reagents used to reveal the presence of the bound probe or other SNP detection reagents. The kit can optionally further comprise compartments and/or reagents for, for example, nucleic acid amplification or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art (see, e.g., Weigl et al., “Lab-on-a-chip for drug development”, Adv Drug Deliv Rev. 2003 Feb. 24; 55(3):349-77). In such microfluidic devices, the containers may be referred to as, for example, microfluidic “compartments”, “chambers”, or “channels”.

Microfluidic devices, which may also be referred to as “lab-on-a-chip” systems, biomedical micro-electro-mechanical systems (bioMEMs), or multicomponent integrated systems, are exemplary kits/systems of the present invention for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more SNPs of the present invention. One example of a microfluidic system is disclosed in U.S. Pat. No. 5,589,136, which describes the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. No. 6,153,073, Dubrow et al., and U.S. Pat. No. 6,156,181, Parce et al.

For genotyping SNPs, a microfluidic system may integrate, for example, nucleic acid amplification, primer extension, capillary electrophoresis, and a detection method such as laser induced fluorescence detection.

Apparatus for Using Nucleic Acid Molecules

The present invention further provides an apparatus for detecting scoliosis mutations comprising a DNA chip array comprising a plurality of polynucleotides attached to the array, wherein each polynucleotide contains a polymorphism selected from the group consisting of the polymorphisms set forth in Table 1 or a polymorphism that is in linkage disequilibrium with a polymorphism of Table 1 or a complement thereof, and a device for detecting the SNPs.

The polymorphism may be selected from the polymorphisms of Table 1. The polymorphism that is in linkage disequilibrium with a polymorphism of Table 1 is selected from the polymorphisms of Tables 2-203.

Uses of Nucleic Acid Molecules

The nucleic acid molecules of the present invention have a variety of uses, especially in the diagnosis and treatment of scoliosis. For example, the nucleic acid molecules are useful as hybridization probes, such as for genotyping SNPs in messenger RNA, transcript, cDNA, genomic DNA, amplified DNA or other nucleic acid molecules disclosed in Table 1 or SNPs disclosed in Tables 1-203, as well as their orthologs.

A probe can hybridize to any nucleotide sequence along the entire length of a nucleic acid molecule encompassing a SNP of the present invention. Preferably, a probe of the present invention hybridizes to a region of a target sequence that encompasses a SNP. More preferably, a probe hybridizes to a SNP-containing target sequence in a sequence- specific manner such that it distinguishes the target sequence from other nucleotide sequences which vary from the target sequence only by which nucleotide is present at the SNP site. Such a probe is particularly useful for detecting the presence of a SNP-containing nucleic acid in a test sample, or for determining which nucleotide (allele) is present at a particular SNP site (i.e., genotyping the SNP site).

A nucleic acid hybridization probe may be used for determining the presence, level, form, and/or distribution of nucleic acid expression. The nucleic acid whose level is determined can be DNA or RNA. Accordingly, probes specific for the SNPs described herein can be used to assess the presence, expression and/or gene copy number in a given cell, tissue, or organism. These uses are relevant for diagnosis of disorders involving an increase or decrease in gene expression relative to normal levels. In vitro techniques for detection of mRNA include, for example, Northern blot hybridizations and in situ hybridizations. In vitro techniques for detecting DNA include Southern blot hybridizations and in situ hybridizations (Sambrook and Russell, 2000, Molecular Cloning: A Laboratory Manual, Cold Spring Harbor Press, Cold Spring Harbor, N.Y.).

Probes can be used as part of a diagnostic test kit for identifying cells or tissues in which a variant protein is expressed, such as by measuring the level of a variant protein-encoding nucleic acid (e.g., mRNA) in a sample of cells from a subject or determining if a polynucleotide contains a SNP of interest.

Thus, the nucleic acid molecules of the invention can be used as hybridization probes to detect the SNPs disclosed herein, thereby determining whether an individual with the polymorphisms is at risk for scoliosis or has developed early stage scoliosis. Detection of a SNP associated with a scoliosis phenotype provides a diagnostic and/or a prognostic tool for an active scoliosis and/or genetic predisposition to the scoliosis.

The nucleic acid molecules of the invention are also useful as primers to amplify any given region of a nucleic acid molecule, particularly a region containing a SNP of the present invention.

The nucleic acid molecules of the invention are also useful for constructing vectors containing a gene regulatory region of the nucleic acid molecules of the present invention.

SNP Genotvping Methods

The process of determining which specific nucleotide (i.e., allele) is present at each of one or more SNP positions, such as a SNP position in a nucleic acid molecule characterized by a SNP of the present invention, is referred to as SNP genotyping. The present invention provides methods of SNP genotyping, such as for use in screening for scoliosis or related pathologies, or determining predisposition thereto, or determining responsiveness to a form of treatment, or in genome mapping or SNP association analysis, etc.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues Mol. Biol. 2003 April; 5(2):43-60; Shi, “Technologies for individual genotyping: detection of genetic polymorphisms in drug targets and scoliosis genes”, Am J Pharmacogenomics. 2002; 2(3):197-205; and Kwok, “Methods for genotyping single nucleotide polymorphisms”, Annu Rev Genomics Hum Genet 2001;2:235-58. Exemplary techniques for high-throughput SNP genotyping are described in Marnellos, “High-throughput SNP analysis for genetic association studies”, Curr Opin Drug Discov Devel. 2003 May; 6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, mass spectrometry with or with monoisotopic dNTPs (U.S. Pat. No. 6,734,294), pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, electrospray mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and S1 protection or chemical cleavage methods.

In a preferred embodiment, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP- containing template which is amplified during PCR, and the probe is designed to hybridize to the target SNP site only if a particular SNP allele is present.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in diagnostic assays for scoliosis and related pathologies, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

Another preferred method for genotyping the SNPs of the present invention is the use of two oligonucleotide probes in an OLA (see, e.g., U.S. Pat. No. 4,988,617). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of OLA: U.S. Pat. Nos. 6,027,889, 6,268,148, 5,494,810, 5,830,711, and 6,054,564 describe OLA strategies for performing SNP detection; WO 97/31256 and WO 00/56927 describe OLA strategies for performing SNP detection using universal arrays, wherein a zipcode sequence can be introduced into one of the hybridization probes, and the resulting product, or amplified product, hybridized to a universal zip code array; U.S. application US01/17329 (and Ser. No. 09/584,905) describes OLA (or LDR) followed by PCR, wherein zipcodes are incorporated into OLA probes, and amplified PCR products are determined by electrophoretic or universal zipcode array readout; U.S. application 60/427,818, 60/445,636, and 60/445,494 describe SNPlex methods and software for multiplexed SNP detection using OLA followed by PCR, wherein zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

The following references provide further information describing mass spectrometry-based methods for SNP genotyping: Bocker, “SNP and mutation discovery using base-specific cleavage and MALDI-TOF mass spectrometry”, Bioinformatics. 2003 July; 19 Suppl 1:144-153; Storm et al., “MALDI-TOF mass spectrometry-based SNP genotyping”, Methods Mol. Biol. 2003;212:241-62; Jurinke et al., “The use of MassARRAY technology for high throughput genotyping”, Adv Biochem Eng Biotechnol. 2002;77:57-74; and Jurinke et al., “Automated genotyping using the DNA MassArray technology”, Methods Mol. Biol. 2002; 187:179-92.

An even more preferred method for genotyping the SNPs of the present invention is the use of electrospray mass spectrometry for direct analysis of an amplified nucleic acid (see, e.g., U.S. Pat. No. 6,734,294). In this method, in one aspect, an amplified nucleic acid product may be isotopically enriched in an isotope of oxygen (0), carbon (C), nitrogen (N) or any combination of those elements. In a preferred embodiment the amplified nucleic acid is isotopically enriched to a level of greater than 99.9% in the elements of O¹⁶, C^{12 and} N¹⁴ The amplified isotopically enriched product can then be analyzed by electrospray mass spectrometry to determine the nucleic acid composition and the corresponding SNP genotyping. Isotopically enriched amplified products result in a corresponding increase in sensitivity and accuracy in the mass spectrum. In another aspect of this method an amplified nucleic acid that is not isotopically enriched can also have composition and SNP genotype determined by electrospray mass spectrometry.

SNPs can also be scored by direct DNA sequencing. A variety of automated sequencing procedures can be utilized ((1995) Biotechniques 19:448), including sequencing by mass spectrometry (see, e.g., PCT International Publication No. WO94/16101; Cohen et al., Adv. Chromatogr. 36:127-162 (1996); and Griffin et al., Appl. Biochem. Biotechnol. 38:147-159 (1993)). The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730×1 DNA Analyzers (Foster City, Calif.), is commonly used in the art for automated sequencing.

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

SNP genotyping is useful for numerous practical applications, as described below. Examples of such applications include, but are not limited to, SNP-scoliosis association analysis, scoliosis predisposition screening, scoliosis diagnosis, scoliosis prognosis, scoliosis progression monitoring, determining therapeutic strategies based on an individual's genotype, and stratifying a patient population for clinical trials for a treatment such as minimally invasive device for the treatment of scoliosis.

Analysis of Genetic Association Between SNPs and Phenotypic Traits

SNP genotyping for scoliosis diagnosis, scoliosis predisposition screening, scoliosis prognosis and scoliosis treatment and other uses described herein, typically relies on initially establishing a genetic association between one or more specific SNPs and the particular phenotypic traits of interest.

In a genetic association study, the cause of interest to be tested is a certain allele or a SNP or a combination of alleles or a haplotype from several SNPs. Thus, tissue specimens (e.g., saliva) from the sampled individuals may be collected and genomic DNA genotyped for the SNP(s) of interest. In addition to the phenotypic trait of interest, other information such as demographic (e.g., age, gender, ethnicity, etc.), clinical, and environmental information that may influence the outcome of the trait can be collected to further characterize and define the sample set. Specifically, in a scoliosis genetic association study, information on Cobb angle, Risser sign, age and gender may be collected. In many cases, these factors are known to be associated with diseases and/or SNP allele frequencies. There are likely gene-environment and/or gene-gene interactions as well. Analysis methods to address gene-environment and gene-gene interactions (for example, the effects of the presence of both susceptibility alleles at two different genes can be greater than the effects of the individual alleles at two genes combined) are discussed below.

After all the relevant phenotypic and genotypic information has been obtained, statistical analyses are carried out to determine if there is any significant correlation between the presence of an allele or a genotype with the phenotypic characteristics of an individual. Preferably, data inspection and cleaning are first performed before carrying out statistical tests for genetic association. Epidemiological and clinical data of the samples can be summarized by descriptive statistics with tables and graphs. Data validation is preferably performed to check for data completion, inconsistent entries, and outliers. Chi-squared tests may then be used to check for significant differences between cases and controls for discrete and continuous variables, respectively. To ensure genotyping quality, Hardy-Weinberg disequilibrium tests can be performed on cases and controls separately. Significant deviation from Hardy-Weinberg equilibrium (HWE) in both cases and controls for individual markers can be indicative of genotyping errors. If HWE is violated in a majority of markers, it is indicative of population substructure that should be further investigated. Moreover, Hardy-Weinberg disequilibrium in cases only can indicate genetic association of the markers with the disease of interest. (Genetic Data Analysis, Weir B., Sinauer (1990)).

To test whether an allele of a single SNP is associated with the case or control status of a phenotypic trait, one skilled in the art can compare allele frequencies in cases and controls. Standard chi-squared tests and Fisher exact tests can be carried out on a 2×2 table (2 SNP alleles×2 outcomes in the categorical trait of interest). To test whether genotypes of a SNP are associated, chi-squared tests can be carried out on a 3×2 table (3 genotypes×2 outcomes). Score tests are also carried out for genotypic association to contrast the three genotypic frequencies (major homozygotes, heterozygotes and minor homozygotes) in cases and controls, and to look for trends using 3 different modes of inheritance, namely dominant (with contrast coefficients 2, −1, −1), additive (with contrast coefficients 1, 0, −1) and recessive (with contrast coefficients 1, 1, −2). Odds ratios for minor versus major alleles, and odds ratios for heterozygote and homozygote variants versus the wild type genotypes are calculated with the desired confidence limits, usually 95%.

In order to control for confounding effects and test for interactions is to perform stepwise multiple logistic regression analysis using statistical packages such as SAS or R. Logistic regression is a model-building technique in which the best fitting and most parsimonious model is built to describe the relation between the dichotomous outcome (for instance, getting a certain scoliosis or not) and a set of independent variables (for instance, genotypes of different associated genes, and the associated demographic and environmental factors). The most common model is one in which the logit transformation of the odds ratios is expressed as a linear combination of the variables (main effects) and their cross-product terms (interactions) (Applied Logistic Regression, Hosmer and Lemeshow, Wiley (2000)). To test whether a certain variable or interaction is significantly associated with the outcome, coefficients in the model are first estimated and then tested for statistical significance of their departure from zero.

In addition to performing association tests one marker at a time, haplotype association analysis may also be performed to study a number of markers that are closely linked together. Haplotype association tests can have better power than genotypic or allelic association tests when the tested markers are not the disease-causing mutations themselves but are in linkage disequilibrium with such mutations. The test will even be more powerful if the scoliosis is indeed caused by a combination of alleles on a haplotype. In order to perform haplotype association effectively, marker-marker linkage disequilibrium measures, both D′ and r², are typically calculated for the markers within a gene to elucidate the haplotype structure. Recent studies (Daly et al, Nature Genetics, 29, 232-235, 2001) in linkage disequilibrium indicate that SNPs within a gene are organized in block pattern, and a high degree of linkage disequilibrium exists within blocks and very little linkage disequilibrium exists between blocks. Haplotype association with the scoliosis status can be performed using such blocks once they have been elucidated.

Haplotype association tests can be carried out in a similar fashion as the allelic and genotypic association tests. Each haplotype in a gene is analogous to an allele in a multi-allelic marker. One skilled in the art can either compare the haplotype frequencies in cases and controls or test genetic association with different pairs of haplotypes. It has been proposed (Schaid et al, Am. J. Hum. Genet., 70, 425-434, 2002) that score tests can be done on haplotypes using the program “haplo.score”. In that method, haplotypes are first inferred by EM algorithm and score tests are carried out with a generalized linear model (GLM) framework that allows the adjustment of other factors.

An important decision in the performance of genetic association tests is the determination of the significance level at which significant association can be declared when the p-value of the tests reaches that level. In an exploratory analysis where positive hits will be followed up in subsequent confirmatory testing, an unadjusted p-value <0. 1 (a significance level on the lenient side) may be used for generating hypotheses for significant association of a SNP with certain phenotypic characteristics of a scoliosis. It is preferred that a p-value <0.05 (a significance level traditionally used in the art) is achieved in order for a SNP to be considered to have an association with a scoliosis. It is more preferred that a p-value <0.01 (a significance level on the stringent side) is achieved for an association to be declared. However, in select instances, a SNP having a p-value >0.05 may be declared to have an association for reasons such as having a high diagnostic odds ratio. When hits are followed up in confirmatory analyses in more samples of the same source or in different samples from different sources, adjustment for multiple testing will be performed as to avoid excess number of hits while maintaining the experiment-wise error rates at 0.05. While there are different methods to adjust for multiple testing to control for different kinds of error rates, a commonly used but rather conservative method is Bonferroni correction to control the experiment-wise or family-wise error rate (Multiple comparisons and multiple tests, Westfall et al, SAS Institute (1999)). Permutation tests to control for the false discovery rates, FDR, can be more powerful (Benjamini and Hochberg, Journal of the Royal Statistical Society, Series B 57, 1289-1300, 1995, Resampling-based Multiple Testing, Westfall and Young, Wiley (1993)). Such methods to control for multiplicity would be preferred when the tests are dependent and controlling for false discovery rates is sufficient as opposed to controlling for the experiment-wise error rates.

In replication studies using samples from different populations after statistically significant markers have been identified in the exploratory stage, meta-analyses can then be performed by combining evidence of different studies (Modern Epidemiology, Lippincott Williams & Wilkins, 1998, 643-673). If available, association results known in the art for the same SNPs can be included in the meta-analyses.

Since both genotyping and scoliosis status classification can involve errors, sensitivity analyses may be performed to see how odds ratios and p-values would change upon various estimates on genotyping and scoliosis classification error rates.

Once individual risk factors, genetic or non-genetic, have been found for the predisposition to scoliosis, the next step is to set up a classification/prediction scheme to predict the category (for instance, scoliosis, no scoliosis, progression curve or non- progressive curve) that an individual will be in depending on his genotypes of associated SNPs and other non-genetic risk factors. Logistic regression for discrete trait and linear regression for continuous trait are standard techniques for such tasks (Applied Regression Analysis, Draper and Smith, Wiley (1998)). Moreover, other techniques can also be used for setting up classification. Such techniques include, but are not limited to, MART, CART, neural network, and discriminant analyses that are suitable for use in comparing the performance of different methods (The Elements of Statistical Learning, Hastie, Tibshirani & Friedman, Springer (2002)).

Scoliosis Diagnosis and Predisposition Screening

Information on association/correlation between genotypes and scoliosis-related phenotypes can be exploited in several ways. For example, in the case of a highly statistically significant association between one or more SNPs with predisposition to a disease for which treatment is available, detection of such a genotype pattern in an individual may justify particular treatment, or at least the institution of regular monitoring of the individual. Detection of the susceptibility alleles associated with a disease in a couple contemplating having children may also be valuable to the couple in their reproductive decisions. In the case of a weaker but still statistically significant association between a SNP and a human disease immediate therapeutic intervention or monitoring may not be justified after detecting the susceptibility allele or SNP.

The SNPs of the invention may contribute to scoliosis in an individual in different ways. Some polymorphisms occur within a protein coding sequence and contribute to scoliosis phenotype by affecting protein structure. Other polymorphisms occur in noncoding regions but may exert phenotypic effects indirectly via influence on, for example, replication, transcription, and/or translation. A single SNP may affect more than one phenotypic trait. Likewise, a single phenotypic trait may be affected by multiple SNPs in different genes.

As used herein, the terms “diagnose”, “diagnosis”, and “diagnostics” include, but are not limited to any of the following: detection of scoliosis that an individual may presently have or be at risk for, predisposition screening (i.e., determining the increased risk for an individual in developing scoliosis in the future, or determining whether an individual has a decreased risk of developing scoliosis in the future;), determining a particular type or subclass of scoliosis in an individual known to have scoliosis, confirming or reinforcing a previously made diagnosis of scoliosis, predicting a progression of a curve and evaluating the future prognosis of an individual having scoliosis. Such diagnostic uses are based on the SNPs individually or in a unique combination or SNP haplotypes of the present invention or in combination with SNPs and other non-genetic clinical factors.

Haplotypes are particularly useful in that, for example, fewer SNPs can be genotyped to determine if a particular genomic region harbors a locus that influences a particular phenotype, such as in linkage disequilibrium-based SNP association analysis.

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For diagnostic purposes, if a particular SNP site is found to be useful for diagnosing scoliosis, then the skilled artisan would recognize that other SNP sites which are in LD with this SNP site would also be useful for diagnosing the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For diagnostic applications, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., scoliosis) that is influenced by the causative SNP(s). Thus, polymorphic markers that are in LD with causative polymorphisms are useful as diagnostic markers, and are particularly useful when the actual causative polymorphism(s) is/are unknown.

Linkage disequilibrium in the human genome is reviewed in: Wall et al., “Haplotype blocks and linkage disequilibrium in the human genome”, Nat Rev Genet. 2003 August; 4(8):587-97; Garner et al., “On selecting markers for association studies: patterns of linkage disequilibrium between two and three diallelic loci”, Genet Epidemiol. 2003 January; 24(1):57-67; Ardlie et al., “Patterns of linkage disequilibrium in the human genome”, Nat Rev Genet. 2002 April; 3(4):299-309 (erratum in Nat Rev Genet 2002 July; 3(7):566); and Remm et al., “High-density genotyping and linkage disequilibrium in the human genome using chromosome 22 as a model”; Curr Opin Chem Biol. 2002 February; 6(1):24-30.

The contribution or association of particular SNPs and/or SNP haplotypes with scoliosis phenotypes, such as adolescent idiopathic scoliosis, enables the SNPs of the present invention to be used to develop superior diagnostic tests capable of identifying individuals who express a detectable trait, such as scoliosis. as the result of a specific genotype, or individuals whose genotype places them at an increased or decreased risk of developing a detectable trait at a subsequent time as compared to individuals who do not have that genotype. As described herein, diagnostics may be based on a single SNP or a group of SNPs. Combined detection of a plurality of SNPs (for example, 2, 3, 4, 5, 6, 7, 8, 9, 10, 11, 12, 13, 14, 15, 16, 17, 18, 19,20,24,25,30,32,48,50,64,96, 100,oranyother number in-between, or more, of the SNPs provided in Table 1 typically increases the probability of an accurate diagnosis. For example, the presence of a single SNP known to correlate with scoliosis might indicate a odds ratio of 1.5 that an individual has or is at risk of developing scoliosis, whereas detection of five SNPs, each of which correlates with scoliosis, might indicate an odds ratio of 9.5 that an individual has or is at risk of developing scoliosis. To further increase the accuracy of diagnosis or predisposition screening, analysis of the SNPs of the present invention can be combined with that of other polymorphisms or other risk factors of scoliosis, such as Cobb angle, Risser sign, gender and age.

It will, of course, be understood by practitioners skilled in the treatment or diagnosis of scoliosis that the present invention generally does not intend to provide an absolute identification of individuals who are at risk (or less at risk) of developing scoliosis. and/or pathologies related to scoliosis, but rather to indicate a certain increased (or decreased) degree or likelihood of developing the scoliosis or developing a progressive curve based on statistically significant association results. However, this information is extremely valuable as it can be used to, for example, initiate earlier preventive and/or corrective treatments or to allow an individual carrying one or more significant SNPs or SNP haplotypes to regularly scheduled physical exams to monitor for the appearance or change of their scoliosis in order to identify and begin treatment of the scoliosis at an early stage.

The diagnostic techniques of the present invention may employ a variety of methodologies to determine whether a test subject has a SNP or a SNP pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the invention may be any detectable trait that is commonly observed in pathologies and disorders related to scoliosis.

Another aspect of the present invention relates to a method of determining whether an individual is at risk (or less at risk) of developing one or more traits or whether an individual expresses one or more traits as a consequence of possessing a particular trait-causing or trait-influencing allele. These methods generally involve obtaining a nucleic acid sample from an individual and assaying the nucleic acid sample to determine which nucleotide(s) is/are present at one or more SNP positions, wherein the assayed nucleotide(s) is/are indicative of an increased or decreased risk of developing the trait or indicative that the individual expresses the trait as a result of possessing a particular trait-causing or trait- influencing allele.

The SNPs of the present invention also can be used to identify novel therapeutic targets for scoliosis. For example, genes containing the disease-associated variants (“variant genes”) or their products, as well as genes or their products that are directly or indirectly regulated by or interacting with these variant genes or their products can be targeted for the development of therapeutics that, for example, treat the scoliosis or prevent or delay scoliosis onset. The therapeutics may be composed of, for example, small molecules, proteins, protein fragments or peptides, antibodies, nucleic acids, or their derivatives or mimetics which modulate the functions or levels of the target genes or gene products.

The SNPs/haplotypes of the present invention are also useful for improving many different aspects of the drug development process. For example, individuals can be selected for clinical trials based on their SNP genotype. Individuals with SNP genotypes that indicate that they are most likely to respond to or most likely to benefit from a device or a drug can be included in the trials and those individuals whose SNP genotypes indicate that they are less likely to or would not respond to a device or a drug, or suffer adverse reactions, can be eliminated from the clinical trials. This not only improves the safety of clinical trials, but also will enhance the chances that the trial will demonstrate statistically significant efficacy. Furthermore, the SNPs of the present invention may explain why certain previously developed devices or drugs performed poorly in clinical trials and may help identify a subset of the population that would benefit from a drug that had previously performed poorly in clinical trials, thereby “rescuing” previously developed devices or drugs, and enabling the device or drug to be made available to a particular scoliosis patient population that can benefit from it.

Pharmaceutical Compositions

Any of the scoliosis-associated proteins, and encoding nucleic acid molecules, disclosed herein can be used as therapeutic targets (or directly used themselves as therapeutic compounds) for treating scoliosis and related pathologies, and the present disclosure enables therapeutic compounds (e.g., small molecules, antibodies, therapeutic proteins, RNAi and antisense molecules, etc.) to be developed that target (or are comprised of) any of these therapeutic targets.

Variant Proteins Encoded by SNP-Containing Nucleic Acid Molecules

The present invention provides SNP-containing nucleic acid molecules, many of which encode proteins having variant amino acid sequences as compared to the art- known (i.e., wild-type) proteins. These variants will generally be referred to herein as variant proteins/peptides/polypeptides, or polymorphic proteins/peptides/polypeptides of the present invention. The terms “protein,” “peptide,” and “polypeptide” are used herein interchangeably.

A variant protein of the present invention may be encoded by, for example, a nonsynonymous nucleotide substitution at any one of the cSNP positions disclosed herein. In addition, variant proteins may also include proteins whose expression, structure, and/or function is altered by a SNP disclosed herein, such as a SNP that creates or destroys a stop codon, a SNP that affects splicing, and a SNP in control/regulatory elements, e.g. promoters, enhancers, or transcription factor binding domains.

Uses of Variant Proteins

The variant proteins of the present invention can be used in a variety of ways, including but not limited to, in assays to determine the biological activity of a variant protein, such as in a panel of multiple proteins for high-throughput screening; to raise antibodies or to elicit another type of immune response; as a reagent (including the labeled reagent) in assays designed to quantitatively determine levels of the variant protein (or its binding partner) in biological fluids; as a marker for cells or tissues in which it is preferentially expressed (either constitutively or at a particular stage of tissue differentiation or development or in a scoliosis state); as a target for screening for a therapeutic agent; and as a direct therapeutic agent to be administered into a human subject. Any of the variant proteins disclosed herein may be developed into reagent grade or kit format for commercialization as research products. Methods for performing the uses listed above are well known to those skilled in the art (see, e.g., Molecular Cloning: A Laboratory Manual, Cold Spring Harbor Laboratory Press, Sambrook and Russell, 2000, and Methods in Enzymology: Guide to Molecular Cloning Techniques, Academic Press, Berger, S. L. and A. R. Kimmel eds., 1987).

Computer-Related Embodiments

The SNPs provided in the present invention may be “provided” in a variety of mediums to facilitate use thereof. As used in this section, “provided” refers to a manufacture, other than an isolated nucleic acid molecule, that contains SNP information of the present invention. Such a manufacture provides the SNP information in a form that allows a skilled artisan to examine the manufacture using means not directly applicable to examining the SNPs or a subset thereof as they exist in nature or in purified form. The SNP information that may be provided in such a form includes any of the SNP information provided by the present invention such as, for example, polymorphic nucleic acid and/or amino acid sequence information of Tables 1-203; information about observed SNP alleles, alternative codons, populations, allele frequencies, SNP types, and/or affected proteins; or any other information provided by the present invention in Tables 1-203 and/or the Sequence Listing.

In one application of this embodiment, the SNPs of the present invention can be recorded on a computer readable medium. As used herein, “computer readable medium” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon a nucleotide sequence of the present invention. One such medium is provided with the present application, namely, the present application contains computer readable medium (CD-R) that has nucleic acid sequences (and encoded protein sequences) containing SNPs provided/recorded thereon in ASCII text format in a Sequence Listing along with accompanying Tables that contain detailed SNP and sequence information.

As used herein, “recorded” refers to a process for storing information on computer readable medium. A skilled artisan can readily adopt any of the presently known methods for recording information on computer readable medium to generate manufactures comprising the SNP information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide/amino acid sequence information of the present invention on computer readable medium. For example, the sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the SNP information of the present invention.

By providing the SNPs of the present invention in computer readable form, a skilled artisan can routinely access the SNP information for a variety of purposes. Computer software is publicly available which allows a skilled artisan to access sequence information provided in a computer readable medium. Examples of publicly available computer software include BLAST (Altschul et at, J. Mol. Biol. 215:403-410 (1990)) and BLAZE (Brutlag et at, Comp. Chem. 17:203-207 (1993)) search algorithms.

The present invention further provides systems, particularly computer-based systems, which contain the SNP information described herein. Such systems may be designed to store and/or analyze information on, for example, a large number of SNP positions, or information on SNP genotypes from a large number of individuals. The SNP information of the present invention represents a valuable information source. The SNP information of the present invention stored/analyzed in a computer-based system may be used for such computer-intensive applications as determining or analyzing SNP allele frequencies in a population, mapping scoliosis genes, genotype-phenotype association studies, grouping SNPs into haplotypes, correlating SNP haplotypes with response to particular treatments or for various other bioinformatic, pharmacogenomic or drug development.

As used herein, “a computer-based system” refers to the hardware means, software means, and data storage means used to analyze the SNP information of the present invention. The minimum hardware means of the computer-based systems of the present invention typically comprises a central processing unit (CPU), input means, output means, and data storage means. A skilled artisan can readily appreciate that any one of the currently available computer-based systems are suitable for use in the present invention. Such a system can be changed into a system of the present invention by utilizing the SNP information provided on the CD-R, or a subset thereof, without any experimentation.

As stated above, the computer-based systems of the present invention comprise a data storage means having stored therein SNPs of the present invention and the necessary hardware means and software means for supporting and implementing a search means. As used herein, “data storage means” refers to memory which can store SNP information of the present invention, or a memory access means which can access manufactures having recorded thereon the SNP information of the present invention.

As used herein, “search means” refers to one or more programs or algorithms that are implemented on the computer-based system to identify or analyze SNPs in a target sequence based on the SNP information stored within the data storage means. Search means can be used to determine which nucleotide is present at a particular SNP position in the target sequence. As used herein, a “target sequence” can be any DNA sequence containing the SNP position(s) to be searched or queried.

As used herein, “a target structural motif,” or “target motif,” refers to any rationally selected sequence or combination of sequences containing a SNP position in which the sequence(s) is chosen based on a three-dimensional configuration that is formed upon the folding of the target motif. There are a variety of target motifs known in the art. Protein target motifs include, but are not limited to, enzymatic active sites and signal sequences. Nucleic acid target motifs include, but are not limited to, promoter sequences, hairpin structures, and inducible expression elements (protein binding sequences).

A variety of structural formats for the input and output means can be used to input and output the information in the computer-based systems of the present invention. An exemplary format for an output means is a display that depicts the presence or absence of specified nucleotides (alleles) at particular SNP positions of interest. Such presentation can provide a rapid, binary scoring system for many SNPs simultaneously.

### EXAMPLES

A whole-genome case-control approach was used to identify the single nucleotide polymorphisms of the present invention that are closely associated with the development of idiopathic adolescent scoliosis (AIS) and specifically progression to a surgical curve (Cobb angle >40°). Samples and controls were collected from the same geographical region, were Caucasian and generally of Northern and Western European descent. Individuals were determined to have AIS after medical record and X-ray review by a single orthopedic surgeon. In one example, about 183 DNA samples from scoliosis patients and 94 controls were genotyped using the Affymetrix GeneChip 100K mapping SNP microarray system. Controls were defined as individuals from the same geographical region who did not have scoliosis (Cobb angle <10°).

A SNP is a DNA sequence variation, occurring when a single nucleotide—adenine (A), thymine (T), cytosine (C) or guanine (G)—in the genome differs between individuals. A variation must occur in at least 1% of the population to be considered a SNP. Variations that occur in less than 1% of the population are, by definition considered to be mutations whether they cause disease or not. SNPs make up 90% of all human genetic variations, and occur every 100 to 300 bases along the human genome. On average, two of every three SNPs substitute cytosine (C) with thymine (T).

GeneChip microarrays consist of small DNA fragments (referred to as probes), chemically synthesized at specific locations on a coated quartz surface. The precise location where each probe is synthesized is called a feature, and millions of features can be contained on one array. The probes which represent a sequence known to contain a human SNP were selected by Affymetrix based on reliability, sensitivity and specificity. In addition to these criteria, the probes were selected to cover the human genome at approximately equal intervals.

The Affymetrix GeneChip 100K mapping array consisted of two microarray chips, the XbaI and HindIII chips, with approximately 58,000 SNPs on each array. Briefly, 250 ng of genomic DNA was digested with either XbaI or HindIII restriction endonuclease and digested fragments were ligated to adapters that contain a universal sequence. The ligated products were then amplified using the polymerase chain reaction (PCR) to amplify fragments between 250-2000 bp in length. The PCR products were purified and diluted to a standard concentration. Furthermore, the PCR products were then fragmented with a DNase enzyme to approximately 25-150 bp in length. This fragmentation process further reduced the complexity of the genomic sample. Still further, the fragmented PCR products were labeled with a biotin/streptavidin system and allowed to hybridize to the microarray. After hybridization the arrays were stained and non-specific binding was removed through a series of increasingly stringent washes. The genotypes were determined by detection of the label in an Affymetrix GCS 3000 scanner. Finally, genotypes were automatically called using Affymetrix G-type software.

For the data to be considered valid for an individual chip, two internal quality control measures were used. SNP genotypes must have exceeded an overall call rate of >90% and the correct gender of the sample needed to be determined as based on the heterozygosity of the X chromosome SNPs. Genotypes were analyzed for significance using Haploview software. A SNP that did not have at least an 80% call rate across all subjects was eliminated as having possible genotyping errors. SNPs that were monomorphic, having no apparent variation in cases or controls, were also eliminated from analysis. After removal of these SNPs approximately 106,000 SNPs were available for analysis.

For each SNP, allelic association was tested against disease affection status. To correct for multiple testing a Bonferroni correction factor was applied to indicate a level at which a SNP would be considered significant. In this case P<0.05/106,000=4.7×10⁻⁷ was considered to be significant. Of the SNPs tested, 202 SNPs were determined to have this level of significance (Table 1).

A second set of cases and controls were selected for genotyping some of the SNPs of Table 1 and three SNPs in linkage disequilibrium with SNPs identified in Table 1 as having an association with scoliosis. The total number of tested SNPs was twelve. To eliminate any possibility of a bias in initial patient and control samples, patient and control samples for this analysis were selected from a nationwide collection of Caucasian samples. Medical histories, X-rays and DNA samples of 675 patients were collected from spine centers across the United States. All subjects were adults with the progression of their scoliosis during adolescence documented. 454 of these subjects had scoliosis which had progressed to a “surgical” curve, 34 had curves >25 degrees that stabilized without treatment or with bracing and 187 had mild scoliosis (risk of progression <3% by Lonstein/Carlson criteria. Progression to a surgical curve (n=454) was defined per usual clinical criteria as: progression to a >40° curve in an individual still growing or progression to a >50° curve in an adult. Of the “surgical cases,” 96% had actually had surgery.

Further, genotyping in this set of samples was performed using a Taqman Assay, as described herein, (Applied Biosystems, Inc.) when available. Briefly, these assays used a standard Taqman minor groove binding (MGB) allele discrimination chemistry where two fluorescent dyes act as detectors and a nonfluorescent quencher is used. Data was collected at end of the PCR reaction. These allele discrimination assays used a unique pair of fluorescent dye detectors that target the SNP site. One fluorescent dye was a perfect match to the wild type allele and a different fluorescent dye was a perfect match to the variant allele. The assay is able to discriminate homozygotes of either the wild type or the variant and heterozygotes.

Genotyping in these selected SNPs was completed on the 675 samples for each of these SNPs. Genotypes were determined using an ABI's automated Taqman genotyping software SDS (v2.1). In this experiment those patients with a known surgical curve were compared to patients with mild scoliosis. This approach was used to determine the utility of the tested SNPs in the prediction of progression to a surgical curve. After this genotype analysis, all twelve tested SNPs showed significant differences between the two patient populations, surgical cases versus non-progressive controls, and were determined to have diagnostic utility. All markers have p values less than 0.003 in Chi square contingency analyses of genotype versus progression to surgery.

Diagnostic odds ratios were calculated. A clinical risk score between 0 and 2 (i.e. conservatively weighted) was estimated based on the subject's records from their first radiologic evaluation for scoliosis (blinded to the genetic data). Genotype weighting factors were estimated from the initial independent discovery sample set. A risk of progression score was generated for each individual. Based on this data, 181 of the 187 mild cases were correctly classified as LOW risk (97% correct), 416 of the 454 patients with surgical curves were correctly classified as HIGH risk (92% correct) and 24 subjects were correctly classified as having an intermediate risk. 54 subjects were incorrectly classified (8% incorrect).

Tables

Lengthy table referenced here
US20090035772A1-20090205-T00001
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20090035772A1). An electronic copy of the table will also be available from the USPTO upon request and payment of the fee set forth in 37 CFR 1.19(b)(3).

What is claimed is:

1. A method for determining predisposition of a human subject to a scoliosis related condition, said method comprising detecting in the genetic material of said human subject the presence or absence of at least one polymorphism of the following group of polymorphisms comprising the polymorphisms of Table 1, the polymorphisms in linkage disequilibrium with the polymorphisms of Table 1, a compliment of the polymorphisms of Table 1, a compliment of the polymorphisms in linkage disequilibrium with the polymorphisms of Table 1, and the polymorphisms of Tables 2-203.

2. The method of claim 1, wherein said scoliosis related condition defines at least one scoliosis related condition of the following group of scoliosis related conditions comprising a scoliosis existence condition, a scoliosis nonexistence condition, a risk of developing scoliosis condition, and a risk of scoliosis curve progression condition, wherein said scoliosis existence condition, said risk of developing scoliosis condition, and said risk of scoliosis curve progression condition define a positive scoliosis related condition.

3. The method of claim 2, wherein said at least one polymorphism includes at least one protective polymorphism and at least one high-risk polymorphism, and wherein said high-risk polymorphism is correlated with an increased risk of a positive scoliosis related condition.

4. The method of claim 1 wherein said at least one polymorphism is correlated with said at least one scoliosis condition, and wherein said correlation defines a correlation having a Chi square contingency p value of no more than 0.01 between an altered risk of a scoliosis related condition population and a control population.

5. The method of claim 1 wherein said method includes the step of evaluating at least one scoliosis related clinical factor of said human subject of the following group of scoliosis related clinical factors comprising Cobb angle, age, Risser sign, age at menarche and gender.

6. The method of claim 5, wherein said method further includes the step of designating an assessed risk of predisposition of said human subject to said at least one scoliosis related condition based on the result of said detection step and said evaluation step.

7. The method of claim 1, wherein said human subject is a human fetus.

8. The method of claim 1, wherein said human subject is designated as having an increased risk of predisposition to a positive scoliosis condition, when said human subject exhibits a Cobb angle of at least 10 degrees.

9. The method of claim 1, wherein in response to an assessed increased risk of a positive scoliosis condition, said method further includes at least one step of the following group of steps comprising the step of selecting an appropriate therapeutic that at least partially compensates for a positive scoliosis condition, the step of selecting a recipient of a therapeutic that at least partially compensates for said at least one scoliosis related condition, the step of treating said human subject by administering to said human subject an appropriate therapeutic that at least partially compensates for a positive scoliosis condition, the step of developing an appropriate therapeutic that at least partially compensates for a positive scoliosis condition, and the step of selecting human subjects for clinical trials involving the use of an appropriate therapeutic for treatment of scoliosis.

10. The method of claim 9, wherein said appropriate therapeutic further defines at least one therapeutic of the following group of therapeutics comprising at least one medical device, at least one pharmaceutical, and at least one medical device and at least one pharmaceutical.

11. The method of claim 1, wherein said method further includes the step of assessing scoliosis risk by determining whether each of a set of independent variables has a unique predictive relationship to a dichotomous dependent variable.

12. The method of claim 11, wherein the step of assessing scoliosis risk comprises an algorithm comprising a logistic regression analysis.

13. A method of performing a medical function, said method comprising the following steps:
obtaining a genetic material sample of a human subject, inspecting said genetic material, detecting in said genetic material the presence or absence of at least one polymorphism of the following group of polymorphisms comprising the polymorphisms of Table 1, the polymorphisms in linkage disequilibrium with the polymorphisms of Table 1, a compliment of the polymorphisms of Table 1, a compliment of the polymorphisms in linkage disequilibrium with the polymorphisms of Table 1, and the polymorphisms of Tables 2-203, and
performing at least one process of the following group of processes comprising: designating an assessed risk of predisposition of said human subject to a scoliosis related condition, assessing an altered risk of a scoliosis related condition by determining whether each of a set of independent variables has a unique predictive relationship to a dichotomous dependent variable, selecting an appropriate therapeutic that at least partially compensates for a scoliosis condition, selecting said human subject as a recipient of a therapeutic that at least partially compensates for a scoliosis related condition, treating said human subject by administering to said human subject an appropriate therapeutic that at least partially compensates for a scoliosis condition, developing an appropriate therapeutic that at least partially compensates for a scoliosis condition, selecting said human subject for clinical trials involving the use of an appropriate therapeutic for treatment of a scoliosis condition, and designating said human as having an increased risk of predisposition to a scoliosis condition when said human subject exhibits a Cobb angle of at least 10 degrees.

14. The method of claim 13, wherein said at least one polymorphism is correlated to at least one scoliosis related condition of the following group of scoliosis related conditions comprising a scoliosis existence condition, a scoliosis nonexistence condition, a risk of developing scoliosis condition, and a risk of scoliosis curve progression condition, wherein said scoliosis existence condition, said risk of developing scoliosis condition, and said risk of scoliosis curve progression condition define a positive scoliosis related condition.

15. The method of claim 14, wherein said at least one polymorphism includes at least one protective polymorphism and at least one high-risk polymorphism, and wherein said high-risk polymorphism is correlated with an increased risk of a positive scoliosis related condition.

16. The method of claim 15 wherein said correlation defines a correlation having a Chi square contingency p value of no more than 0.01 between an altered risk of a scoliosis related condition population and a control population.

17. The method of claim 13 wherein said method includes the step of evaluating at least one scoliosis related clinical factor of said human subject of the following group of scoliosis related clinical factors comprising Cobb angle, age, Risser sign, age at menarche and gender.

18. The method of claim 17, wherein said method further includes the step of designating an assessed risk of predisposition of said human subject to said at least one scoliosis related condition based on the result of said detection step and said evaluation step.

19. The method of claim 13, wherein said human subject is a human fetus.

20. The method of claim 13, wherein said appropriate therapeutic further defines at least one therapeutic of the following group of therapeutics comprising at least one medical device, at least one pharmaceutical, and at least one medical device and at least one pharmaceutical.

21. The method of claim 13, wherein said method further includes the step of assessing scoliosis risk by determining whether each of a set of independent variables has a unique predictive relationship to a dichotomous dependent variable.

22. The method of claim 21, wherein the step of assessing scoliosis risk comprises an algorithm comprising a logistic regression analysis.

23. A method of screening human subjects, said method comprising the following steps:
obtaining a genetic material sample of a human subject,
inspecting said genetic material,
detecting in said genetic material the presence or absence of at least one polymorphism of the following group of polymorphisms comprising the polymorphisms of Table 1, the polymorphisms in linkage disequilibrium with the polymorphisms of Table 1, a compliment of the polymorphisms of Table 1, a compliment of the polymorphisms in linkage disequilibrium with the polymorphisms of Table 1, and the polymorphisms of Tables 2-203, and
responding to said detection of said at least one polymorphism.

24. The method of claim 23, wherein said responding step defines at least one responding step of the following group of responding steps comprising designating an assessed risk of predisposition of said human subject to a scoliosis related condition, assessing an altered risk of a scoliosis related condition by determining whether each of a set of independent variables has a unique predictive relationship to a dichotomous dependent variable, selecting an appropriate therapeutic that at least partially compensates for a scoliosis condition, selecting said human subject as a recipient of a therapeutic that at least partially compensates for a scoliosis related condition, treating said human subject by administering to said human subject an appropriate therapeutic that at least partially compensates for a scoliosis condition, developing an appropriate therapeutic that at least partially compensates for a scoliosis condition, selecting said human subject for clinical trials involving the use of an appropriate therapeutic for treatment of a scoliosis condition, and designating said human as having an increased risk of predisposition to a scoliosis condition when said human subject exhibits a Cobb angle of at least 10 degrees.

25. The method of claim 23, wherein said at least one polymorphism is correlated to at least one scoliosis related condition of the following group of scoliosis related conditions comprising a scoliosis existence condition, a scoliosis nonexistence condition, a risk of developing scoliosis condition, and a risk of scoliosis curve progression condition, wherein said scoliosis existence condition, said risk of developing scoliosis condition, and said risk of scoliosis curve progression condition define a positive scoliosis related condition.

26. The method of claim 25, wherein said at least one polymorphism includes at least one protective polymorphism and at least one high-risk polymorphism, and wherein said high-risk polymorphism is correlated with an increased risk of a positive scoliosis related condition.

27. The method of claim 25 wherein said correlation defines a correlation having a Chi square contingency p value of no more than 0.01 between an altered risk of a scoliosis related condition population and a control population.

28. The method of claim 23 wherein said method includes the step of evaluating at least one scoliosis related clinical factor of said human subject of the following group of scoliosis related clinical factors comprising Cobb angle, age, Risser sign, age at menarche and gender.

29. The method of claim 28, wherein said method further includes the step of designating an assessed risk of predisposition of said human subject to said at least one scoliosis related condition based on the result of said detection step and said evaluation step.

30. The method of claim 23, wherein said human subject is a human fetus.

31. The method of claim 23, wherein said human subject is designated as having an increased risk of predisposition to a positive scoliosis condition, when said human subject exhibits a Cobb angle of at least 10 degrees.

32. The method of claim 24, wherein said appropriate therapeutic further defines at least one therapeutic of the following group of therapeutics comprising at least one medical device, at least one pharmaceutical, and at least one medical device and at least one pharmaceutical.

33. The method of claim 23, wherein said method further includes the step of assessing scoliosis risk by determining whether each of a set of independent variables has a unique predictive relationship to a dichotomous dependent variable.

34. The method of claim 33, wherein the step of assessing scoliosis risk comprises an algorithm comprising a logistic regression analysis.

35. A method of detecting in a nucleic acid molecule a polymorphism that is correlated with a scoliosis related condition, comprising:
contacting a test sample with a polynucleotide sequence that specifically hybridizes under stringent hybridization conditions to a polynucleotide sequence having at least one protective or high-risk polymorphism selected from the group comprising of the polymorphisms of Table 1, the polymorphisms in linkage disequilibrium with the polymorphisms of Table 1, a compliment of the polymorphisms of Table 1, a compliment of the polymorphisms in linkage disequilibrium with the polymorphisms of Table 1, and the polymorphisms of Tables 2-203, and
detecting the formation of a hybridized duplex.

36. The method of claim 35, wherein said polymorphism is correlated with at least one risk of the following group of risks comprising a presymptomatic risk of developing scoliosis in a human subject, and an increased risk of scoliosis curve progression in a human subject having a scoliosis curve.

37. The method of claim 36, wherein said human subject is a human fetus.

38. The method of claim 35, wherein said scoliosis related condition defines at least one scoliosis related condition of the following group of scoliosis related conditions comprising a scoliosis existence condition, a scoliosis nonexistence condition, a risk of developing scoliosis condition, and a risk of scoliosis curve progression condition, wherein said scoliosis existence condition, said risk of developing scoliosis condition, and said risk of scoliosis curve progression condition define a positive scoliosis related condition.

39. The method of claim 35, wherein said method further includes at least one step of the following group of steps comprising the step of selecting an appropriate therapeutic that at least partially compensates for a positive scoliosis condition, the step of selecting a recipient of a therapeutic that at least partially compensates for said at least one scoliosis related condition, the step of treating said human subject by administering to said human subject an appropriate therapeutic that at least partially compensates for a positive scoliosis condition, the step of developing an appropriate therapeutic that at least partially compensates for a positive scoliosis condition, and the step of selecting human subjects for clinical trials involving the use of an appropriate therapeutic for treatment of scoliosis.

40. The method of claim 39, wherein said appropriate therapeutic further defines at least one therapeutic of the following group of therapeutics comprising at least one medical device, at least one pharmaceutical, and at least one medical device and at least one pharmaceutical.

41. The method of claim 35, wherein said method further includes the step of assessing scoliosis risk by determining whether each of a set of independent variables has a unique predictive relationship to a dichotomous dependent variable.

42. The method of claim 41, wherein said step of assessing scoliosis risk comprises an algorithm comprising a logistic regression analysis.

43. A kit for detecting in a subject a nucleic acid polymorphism indicative of an altered risk of a scoliosis related condition, said kit comprising enzymes, buffers, reagents used to detect genetic polymorphisms, and an isolated polynucleotide that specifically hybridizes to a polynucleotide molecule containing the nucleotide sequence of a polymorphism selected from the polymorphisms of Table 1, the polymorphisms in linkage disequilibrium with the polymorphisms of Table 1, a compliment of the polymorphisms of Table 1, a compliment of the polymorphisms in linkage disequilibrium with the polymorphisms of Table 1, and the polymorphisms of Tables 2-203.

44. The method of claim 43, wherein said scoliosis related condition defines at least one scoliosis related condition of the following group of scoliosis related conditions comprising a scoliosis existence condition, a scoliosis nonexistence condition, a risk of developing scoliosis condition, and a risk of scoliosis curve progression condition, wherein said scoliosis existence condition, said risk of developing scoliosis condition, and said risk of scoliosis curve progression condition define a positive scoliosis related condition.

45. The kit of claim 44, wherein said kit further includes a questionnaire of non- genetic clinical factors.

46. An isolated polynucleotide that specifically hybridizes to a polynucleotide molecule containing the nucleotide sequence of a polymorphism selected from the polymorphisms of Table 1, the polymorphisms in linkage disequilibrium with the polymorphisms of Table 1, a compliment of the polymorphisms of Table 1, a compliment of the polymorphisms in linkage disequilibrium with the polymorphisms of Table 1, and the polymorphisms of Tables 2-203.

47. The polynucleotide of claim 46, wherein said polynucleotide is about 8 to 70 nucleotides in length.

48. The polynucleotide of claim 46, wherein said polynucleotide defines one of an allele-specific probe and an allele-specific primer.

49. An amplified polynucleotide containing the nucleotide sequence of a polymorphism selected from the polymorphisms of Table 1, the polymorphisms in linkage disequilibrium with the polymorphisms of Table 1, a compliment of the polymorphisms of Table 1, a compliment of the polymorphisms in linkage disequilibrium with the polymorphisms of Table 1, and the polymorphisms of Tables 2-203, wherein said amplified polynucleotide is greater than about 16 nucleotides in length.

50. An apparatus for detecting scoliosis mutations comprising:
a DNA chip array comprising a plurality of polynucleotides attached to said array, wherein each of said polynucleotides contains a polymorphism selected from the group consisting of the polymorphisms of Table 1, the polymorphisms in linkage disequilibrium with the polymorphisms of Table 1, a compliment of the polymorphisms of Table 1, a compliment of the polymorphisms in linkage disequilibrium with the polymorphisms of Table 1, and the polymorphisms of Tables 2-203,
and a device for detecting said scoliosis mutations.


SEQUENCE LISTING (202 sequences)

SEQ ID NO: 1 - DNA, 33 bp, Homo sapiens
ggggcaagagagacaarcttcggctctgagaac

SEQ ID NO: 2 - DNA, 33 bp, Homo sapiens
ggtcattgggcaatacracattacaagatgctc

SEQ ID NO: 3 - DNA, 33 bp, Homo sapiens
gtaaagacacaaggccrcttatgtttattgcag

SEQ ID NO: 4 - DNA, 33 bp, Homo sapiens
gtagagaaaatttcacyggactctaagagagca

SEQ ID NO: 5 - DNA, 33 bp, Homo sapiens
gtagtgggacaccagcygaccgttgattcatat

SEQ ID NO: 6 - DNA, 33 bp, Homo sapiens
taacatccatgcgaagraaatggaacacttata

SEQ ID NO: 7 - DNA, 33 bp, Homo sapiens
tacccacaactgtaaartgagagagaccgactt

SEQ ID NO: 8 - DNA, 33 bp, Homo sapiens
taggttgattttcccarcaactcactttgaggg

SEQ ID NO: 9 - DNA, 33 bp, Homo sapiens
tcatgcgttttatactsaaagtctgccctgtag

SEQ ID NO: 10 - DNA, 33 bp, Homo sapiens
tccagctacactcaacrcatttcaccccacccc

SEQ ID NO: 11 - DNA, 33 bp, Homo sapiens
ttgcgattctatctgtraagctctgagaatgag

SEQ ID NO: 12 - DNA, 33 bp, Homo sapiens
taaagtcatgtagtacraaaagatgaaaagcct

SEQ ID NO: 13 - DNA, 33 bp, Homo sapiens
tcccatctgagggctcygaaaaaaatgtaaggt

SEQ ID NO: 14 - DNA, 33 bp, Homo sapiens
tcctttagcaagacccrctgcagaagcctgtgc

SEQ ID NO: 15 - DNA, 33 bp, Homo sapiens
tctgctatgaagcttasaggatattatgtgtct

SEQ ID NO: 16 - DNA, 33 bp, Homo sapiens
tctggaatgtggtcctmactgcaaatgttccag

SEQ ID NO: 17 - DNA, 33 bp, Homo sapiens
tgaatgtaatcactcartaaggtttaagatgca

SEQ ID NO: 18 - DNA, 33 bp, Homo sapiens
tgcaagttcaacggaaragcattgattgaccag

SEQ ID NO: 19 - DNA, 33 bp, Homo sapiens
tgccctttctgaaagcsaaattactacaaagat

SEQ ID NO: 20 - DNA, 33 bp, Homo sapiens
tgctcactctgagtcayggtgaaaatgtttcag

SEQ ID NO: 21 - DNA, 33 bp, Homo sapiens
tggaagcttgggagggycaatttagttaaacaa

SEQ ID NO: 22 - DNA, 33 bp, Homo sapiens
tggctcaggcaaggatkagtaagacagcaggac

SEQ ID NO: 23 - DNA, 33 bp, Homo sapiens
tggttgtcactgaaaamgtaagttatgcattcc

SEQ ID NO: 24 - DNA, 33 bp, Homo sapiens
tgtataaccttctgtgmatcaaatagcaacaga

SEQ ID NO: 25 - DNA, 33 bp, Homo sapiens
tgtgattatgaggaaargtgagaataattctat

SEQ ID NO: 26 - DNA, 33 bp, Homo sapiens
tgttctatatgatttayggacataacagcccac

SEQ ID NO: 27 - DNA, 33 bp, Homo sapiens
ttaagataattatgtcrccatcaggatactggt

SEQ ID NO: 28 - DNA, 33 bp, Homo sapiens
ttaagtcacttacccaragcgatacagctggta

SEQ ID NO: 29 - DNA, 33 bp, Homo sapiens
ttagcattatctaaacrggaatatttgggtagc

SEQ ID NO: 30 - DNA, 33 bp, Homo sapiens
ttaggcatactaatcasaaagtccctctgtttt

SEQ ID NO: 31 - DNA, 33 bp, Homo sapiens
ttcatgagcatcttagraggatcacttttccag

SEQ ID NO: 32 - DNA, 33 bp, Homo sapiens
ttgggttctcatttaaytctcccagtatctctg

SEQ ID NO: 33 - DNA, 33 bp, Homo sapiens
tttgatactaaagttaygaaatgcagaagtcaa

SEQ ID NO: 34 - DNA, 33 bp, Homo sapiens
ttttatgaaatacaacygaatttattcttgcat

SEQ ID NO: 35 - DNA, 33 bp, Homo sapiens
ttttcattgaaactacrgcaaagccatacacat

SEQ ID NO: 36 - DNA, 33 bp, Homo sapiens
ttttgtatgttttagakatcacataggtatata

SEQ ID NO: 37 - DNA, 33 bp, Homo sapiens
taatctctggaggtagmaaccattgcttcttcg

SEQ ID NO: 38 - DNA, 33 bp, Homo sapiens
ataggctcaattctttmaaaaatccattgatac

SEQ ID NO: 39 - DNA, 33 bp, Homo sapiens
aaagggccaagaaaaartaactgagaagatgca

SEQ ID NO: 40 - DNA, 33 bp, Homo sapiens
acctggaatgtcaaagrtaaagtgcaacacatt

SEQ ID NO: 41 - DNA, 33 bp, Homo sapiens
ttgctcattgaatataycctgctggctactact

SEQ ID NO: 42 - DNA, 33 bp, Homo sapiens
ttgacaagccctaaccrgctgcatggcattaga

SEQ ID NO: 43 - DNA, 33 bp, Homo sapiens
aacaaatgaccacttaygtatttgagcaaacaa

SEQ ID NO: 44 - DNA, 33 bp, Homo sapiens
tgtgcatgaaagtgtayaatccacagagtaaaa

SEQ ID NO: 45 - DNA, 33 bp, Homo sapiens
tgaacaatcggtggaartcaactgtacacatgt

SEQ ID NO: 46 - DNA, 33 bp, Homo sapiens
gactgtcagggacggcractctttgtccaccgc

SEQ ID NO: 47 - DNA, 33 bp, Homo sapiens
aaatttagattgaagasaaaaaaatcaagaaac

SEQ ID NO: 48 - DNA, 33 bp, Homo sapiens
aacacaagctgcaaaaygcttaacccaggctgg

SEQ ID NO: 49 - DNA, 33 bp, Homo sapiens
aaccaattttttcccamctgatgctgtcatcta

SEQ ID NO: 50 - DNA, 33 bp, Homo sapiens
aatcgtctacatatcargaattagagcattagt

SEQ ID NO: 51 - DNA, 33 bp, Homo sapiens
actagacacaatagaaractacaatagaaggga

SEQ ID NO: 52 - DNA, 33 bp, Homo sapiens
acttcctccttaggtcycaacacaggcgactcc

SEQ ID NO: 53 - DNA, 33 bp, Homo sapiens
tcaaagcaaacattaawtggctgccgttactgc

SEQ ID NO: 54 - DNA, 33 bp, Homo sapiens
agaggctctttgtttcwcacaggaattagtaga

SEQ ID NO: 55 - DNA, 33 bp, Homo sapiens
agatgtgagtgaggaaygatgccatatcctaga

SEQ ID NO: 56 - DNA, 33 bp, Homo sapiens
agctataggcttaatarcaaatgtcctgccctc

SEQ ID NO: 57 - DNA, 33 bp, Homo sapiens
aggaaaatggctctgawtcaactgtagagtcca

SEQ ID NO: 58 - DNA, 33 bp, Homo sapiens
aggggaaagtggctgcracagtatcagagagta

SEQ ID NO: 59 - DNA, 33 bp, Homo sapiens
ataaagcaatgaagccrctaaagatttaatctg

SEQ ID NO: 60 - DNA, 33 bp, Homo sapiens
caaggccagtgagtaartaccaggtccgtgtgc

SEQ ID NO: 61 - DNA, 33 bp, Homo sapiens
caaggtgggggccgaaygatcccaacacagtgg

SEQ ID NO: 62 - DNA, 33 bp, Homo sapiens
cacacatccacacttayaccaaattattaatgt

SEQ ID NO: 63 - DNA, 33 bp, Homo sapiens
caccacctgaggtttcrctatctgcactctgct

SEQ ID NO: 64 - DNA, 33 bp, Homo sapiens
cacctaggcgaatatawacaagactcaaaagca

SEQ ID NO: 65 - DNA, 33 bp, Homo sapiens
cacctcccaggtcctcratgtttggatctgcgt

SEQ ID NO: 66 - DNA, 33 bp, Homo sapiens
cattcgccatctaccarattttcacaacaacct

SEQ ID NO: 67 - DNA, 33 bp, Homo sapiens
cattgccatcatttagragcttgttaagaatga

SEQ ID NO: 68 - DNA, 33 bp, Homo sapiens
catttctttgctttccragtctggatttagagg

SEQ ID NO: 69 - DNA, 33 bp, Homo sapiens
cgaaccatgatccccartgatccactttaggga

SEQ ID NO: 70 - DNA, 33 bp, Homo sapiens
ctcacctgtgttctggyattaactctgcattag

SEQ ID NO: 71 - DNA, 33 bp, Homo sapiens
ctgttgtactttccctyaacattccactttctc

SEQ ID NO: 72 - DNA, 33 bp, Homo sapiens
gaaggtggcaccaccasaatttgctgagtgatt

SEQ ID NO: 73 - DNA, 33 bp, Homo sapiens
gactgtatcttttggtwacagacagacttctga

SEQ ID NO: 74 - DNA, 33 bp, Homo sapiens
gagaagcacagtaagargcaatggtgggcccaa

SEQ ID NO: 75 - DNA, 33 bp, Homo sapiens
gagcatttactggagaytaagccttgtttcaag

SEQ ID NO: 76 - DNA, 33 bp, Homo sapiens
gataacaagtcacatcsaagctgggattcaaac

SEQ ID NO: 77 - DNA, 33 bp, Homo sapiens
gcagggtgttggtaaascacaacggatagtgca

SEQ ID NO: 78 - DNA, 33 bp, Homo sapiens
gcagtgttacagacatkaaggaagaagaaaatg

SEQ ID NO: 79 - DNA, 33 bp, Homo sapiens
gccatgaagctattcaragggcaacagaaatgc

SEQ ID NO: 80 - DNA, 33 bp, Homo sapiens
gctaagatctttcagaraaggacaaagaaatac

SEQ ID NO: 81 - DNA, 33 bp, Homo sapiens
ggaccttagaagatatragcaaaactgcctaac

SEQ ID NO: 82 - DNA, 33 bp, Homo sapiens
ggagctaccaacagtaragccccaacaatcagc

SEQ ID NO: 83 - DNA, 33 bp, Homo sapiens
tgaagagaatattgacygggtgcttccaaaagg

SEQ ID NO: 84 - DNA, 33 bp, Homo sapiens
tcagttgaagatcacayaagctaagagtggcgc

SEQ ID NO: 85 - DNA, 33 bp, Homo sapiens
ttctggaaaagtgcaayaatggcaattctcgta

SEQ ID NO: 86 - DNA, 33 bp, Homo sapiens
tttgtaatgaagggaaygcctatgttctctgcc

SEQ ID NO: 87 - DNA, 33 bp, Homo sapiens
cggaggtgtttgaagamctggttgagagggtct

SEQ ID NO: 88 - DNA, 33 bp, Homo sapiens
tgatatttggttgggawcgtgttttaatagaga

SEQ ID NO: 89 - DNA, 33 bp, Homo sapiens
actgtaacagagaccaytagaacacagctcagg

SEQ ID NO: 90 - DNA, 33 bp, Homo sapiens
aggaatcatttctaaayagcatcttgttattag

SEQ ID NO: 91 - DNA, 33 bp, Homo sapiens
atcagtctctagcatgractgatgcagagtagg

SEQ ID NO: 92 - DNA, 33 bp, Homo sapiens
ccagtgctggaagaaarcaactttactgaggag

SEQ ID NO: 93 - DNA, 33 bp, Homo sapiens
ctctagttcctcatttwaaaattgtcaaatact

SEQ ID NO: 94 - DNA, 33 bp, Homo sapiens
gaacactgcacactaayagggcagactgactga

SEQ ID NO: 95 - DNA, 33 bp, Homo sapiens
ggctttttcaaaggctwacagagtcaaaggaat

SEQ ID NO: 96 - DNA, 33 bp, Homo sapiens
tccgcccgtagacatgraggaatcttctggtgg

SEQ ID NO: 97 - DNA, 33 bp, Homo sapiens
tttctaaggaatgtgargaatcttactctttcc

SEQ ID NO: 98 - DNA, 33 bp, Homo sapiens
gatacataagtcatggyacttgttcctctgggc

SEQ ID NO: 99 - DNA, 33 bp, Homo sapiens
atgtcttgcagatatgrttatagagaaaagagc

SEQ ID NO: 100 - DNA, 33 bp, Homo sapiens
acattaatggatgaacrgaacattgcccatttg

SEQ ID NO: 101 - DNA, 33 bp, Homo sapiens
agcccactgaggcatayagaaaaaagcaatgta

SEQ ID NO: 102 - DNA, 33 bp, Homo sapiens
cacagtcctgtctaaaygtttatttgtcatttt

SEQ ID NO: 103 - DNA, 33 bp, Homo sapiens
cagtgggaaataattargcattttcacgattct

SEQ ID NO: 104 - DNA, 33 bp, Homo sapiens
ccagagctctcagtgcrgacaacagaggccttg

SEQ ID NO: 105 - DNA, 33 bp, Homo sapiens
cctcagaaatgagctcrcaaaatgttctgtact

SEQ ID NO: 106 - DNA, 33 bp, Homo sapiens
ccttgtcttcaaaggaygactctgactgctcca

SEQ ID NO: 107 - DNA, 33 bp, Homo sapiens
ctggcaagtgccataarggaagaaaatagcacc

SEQ ID NO: 108 - DNA, 33 bp, Homo sapiens
gcatcatcagtgttacrgggaagtcactagaaa

SEQ ID NO: 109 - DNA, 33 bp, Homo sapiens
gccacatcttctctgastatcaattgatgaaga

SEQ ID NO: 110 - DNA, 33 bp, Homo sapiens
gcccagaaaaggtgcayataatttgcttccaat

SEQ ID NO: 111 - DNA, 33 bp, Homo sapiens
ggctgagaattcttcamattcctgttttgtttt

SEQ ID NO: 112 - DNA, 33 bp, Homo sapiens
ggctgtattctcagaayggactgagaggccaag

SEQ ID NO: 113 - DNA, 33 bp, Homo sapiens
ggtattgggtaaacacmgctcttccaaatggga

SEQ ID NO: 114 - DNA, 33 bp, Homo sapiens
ggttactcttcatctckccaacctcttaccatt

SEQ ID NO: 115 - DNA, 33 bp, Homo sapiens
gtaaatcatgctgaagkcgactctttaatgatg

SEQ ID NO: 116 - DNA, 33 bp, Homo sapiens
ggttcactttatgaagyctcagctgtttcgggg

SEQ ID NO: 117 - DNA, 33 bp, Homo sapiens
ggtgatggctgtacaaygatgtgagtgaactca

SEQ ID NO: 118 - DNA, 33 bp, Homo sapiens
gtgctctcctggcccaragaaaaataatgctgg

SEQ ID NO: 119 - DNA, 33 bp, Homo sapiens
gtatttgcatagaaccyatacacatcctcccac

SEQ ID NO: 120 - DNA, 33 bp, Homo sapiens
gtgcccctggatacatkatgaaacgtctctgtg

SEQ ID NO: 121 - DNA, 33 bp, Homo sapiens
gtccataccaaaatagycatactgtcagctgga

SEQ ID NO: 122 - DNA, 33 bp, Homo sapiens
gtgggaatagtttcacsagtagttccgagtttg

SEQ ID NO: 123 - DNA, 33 bp, Homo sapiens
tcctcttcccaccagcrgctgatgcgcttttca

SEQ ID NO: 124 - DNA, 33 bp, Homo sapiens
aaatctcaaaagctcayctacctctaagtcagt

SEQ ID NO: 125 - DNA, 33 bp, Homo sapiens
aaaatacttattattayggtggtagtgatattg

SEQ ID NO: 126 - DNA, 33 bp, Homo sapiens
aaaatcaaaagtatctraataccagcatataaa

SEQ ID NO: 127 - DNA, 33 bp, Homo sapiens
aagaagcagctcaccawacaagtgatttaggat

SEQ ID NO: 128 - DNA, 33 bp, Homo sapiens
aacagtaaaattccaaycttgtcatactgcttt

SEQ ID NO: 129 - DNA, 33 bp, Homo sapiens
aaggtagtttgtgaatyaggagcctgggaggaa

SEQ ID NO: 130 - DNA, 33 bp, Homo sapiens
ttcactaacatattccratctgctctcagaatt

SEQ ID NO: 131 - DNA, 33 bp, Homo sapiens
aattaaaaataatgtaygaccctcgattggatc

SEQ ID NO: 132 - DNA, 33 bp, Homo sapiens
acacagagcaagatgcygatgctgaaaatggcc

SEQ ID NO: 133 - DNA, 33 bp, Homo sapiens
tatttgtagagcaaacwcctatccatttttctg

SEQ ID NO: 134 - DNA, 33 bp, Homo sapiens
tatgctggcctcgtacscctcgatgtcccttct

SEQ ID NO: 135 - DNA, 33 bp, Homo sapiens
tattatccccctctcasagctgaagcttccaag

SEQ ID NO: 136 - DNA, 33 bp, Homo sapiens
tcagcttaatggaatasagggcagatttcaggt

SEQ ID NO: 137 - DNA, 33 bp, Homo sapiens
ggccaagggacagacgraagatgaggaggtaaa

SEQ ID NO: 138 - DNA, 33 bp, Homo sapiens
tccttgctcattctgcyccaactacgctggcct

SEQ ID NO: 139 - DNA, 33 bp, Homo sapiens
tgagacctcatctctaygaagaaactgaaacat

SEQ ID NO: 140 - DNA, 33 bp, Homo sapiens
tgacttttgcggggcartactcagcaaccagga

SEQ ID NO: 141 - DNA, 33 bp, Homo sapiens
tgaactagcctgggacygtaaaagaacaggaaa

SEQ ID NO: 142 - DNA, 33 bp, Homo sapiens
tggaagtggagtccaargctgtgaagcttctat

SEQ ID NO: 143 - DNA, 33 bp, Homo sapiens
tgtacaaagccggagcygcagaaaaataacatc

SEQ ID NO: 144 - DNA, 33 bp, Homo sapiens
tgtctaccttcagtaaygtcgttctttttcacc

SEQ ID NO: 145 - DNA, 33 bp, Homo sapiens
acgtggatcatatcaayagacattagaaatcaa

SEQ ID NO: 146 - DNA, 33 bp, Homo sapiens
agaattgtctgaaccartgattaaattcaagag

SEQ ID NO: 147 - DNA, 33 bp, Homo sapiens
agaaacacgctgcatawcatgagaatggaggag

SEQ ID NO: 148 - DNA, 33 bp, Homo sapiens
agaagtaggagaggcckaatctgggtccctagg

SEQ ID NO: 149 - DNA, 33 bp, Homo sapiens
agatgccagatgttccratgggacattgagtta

SEQ ID NO: 150 - DNA, 33 bp, Homo sapiens
aggcaattctgctaacracaaattctctccatt

SEQ ID NO: 151 - DNA, 33 bp, Homo sapiens
agctttacaagaaacakgcctcacttctgcctt

SEQ ID NO: 152 - DNA, 33 bp, Homo sapiens
agattgagaaccagtaygctagtctcagcagta

SEQ ID NO: 153 - DNA, 33 bp, Homo sapiens
agggttcagacctcatyagaaaagtttagttac

SEQ ID NO: 154 - DNA, 33 bp, Homo sapiens
agggtacatccttcacyactatcaaatactgga

SEQ ID NO: 155 - DNA, 33 bp, Homo sapiens
tgtgatataaggactaraattaaggctctgtgt

SEQ ID NO: 156 - DNA, 33 bp, Homo sapiens
ttaaacttagttgaccraagcacccttttcctg

SEQ ID NO: 157 - DNA, 33 bp, Homo sapiens
tgtggcacccacggaarcttctaggcctcatga

SEQ ID NO: 158 - DNA, 33 bp, Homo sapiens
ttccactgtcttgaggwatagcacctgactccc

SEQ ID NO: 159 - DNA, 33 bp, Homo sapiens
ttcttggtgagaacaaygggtcctcctgctgtg

SEQ ID NO: 160 - DNA, 33 bp, Homo sapiens
tttaggaagcaaaaacrgtgcttatcttacccc

SEQ ID NO: 161 - DNA, 33 bp, Homo sapiens
ttctttctcttctaccrcagagtcatctcctag

SEQ ID NO: 162 - DNA, 33 bp, Homo sapiens
ttgtattgatgtaggcrctgcccagtcagcatt

SEQ ID NO: 163 - DNA, 33 bp, Homo sapiens
tttgatataaggcttgyatgtttagagactaac

SEQ ID NO: 164 - DNA, 33 bp, Homo sapiens
tttcctgccttcctacratatgtaatcaactcc

SEQ ID NO: 165 - DNA, 33 bp, Homo sapiens
agtgcaagcagatacarcgtgtggtgcatttga

SEQ ID NO: 166 - DNA, 33 bp, Homo sapiens
agtcttcatgggaagayatgagcagttccctcc

SEQ ID NO: 167 - DNA, 33 bp, Homo sapiens
atactaccagaccttayggacgtttatgttcac

SEQ ID NO: 168 - DNA, 33 bp, Homo sapiens
atcttttcacaattcaratctgattctgctacc

SEQ ID NO: 169 - DNA, 33 bp, Homo sapiens
atgcccctgaagttccygctctaggaaggtgcc

SEQ ID NO: 170 - DNA, 33 bp, Homo sapiens
atgcaagacgttttaaygccaggatgttcgtat

SEQ ID NO: 171 - DNA, 33 bp, Homo sapiens
atggaaggtgggttgcycctagaaagagtgtga

SEQ ID NO: 172 - DNA, 33 bp, Homo sapiens
caacagttatctgtgawattcactttctccctg

SEQ ID NO: 173 - DNA, 33 bp, Homo sapiens
atttacttaactatagwagccattttagtatct

SEQ ID NO: 174 - DNA, 33 bp, Homo sapiens
ctagaaaacaggcttayattttatcataatgag

SEQ ID NO: 175 - DNA, 33 bp, Homo sapiens
atcaagataatgaataygcccacgattctaaaa

SEQ ID NO: 176 - DNA, 33 bp, Homo sapiens
caatcttttcatcaaaygccttttatctcaaca

SEQ ID NO: 177 - DNA, 33 bp, Homo sapiens
cagagagaaactgacawatatacttattccgac

SEQ ID NO: 178 - DNA, 33 bp, Homo sapiens
cagctaccttgacaggracgtccaccttggaag

SEQ ID NO: 179 - DNA, 33 bp, Homo sapiens
cagtaagattaagtaayccgctcacattactta

SEQ ID NO: 180 - DNA, 33 bp, Homo sapiens
ccaggatccctgggccrgattattcgtgattac

SEQ ID NO: 181 - DNA, 33 bp, Homo sapiens
ccaactgactttaaggraaactgtggtatgcag

SEQ ID NO: 182 - DNA, 33 bp, Homo sapiens
catggagccctagtggyatttggtggaggccag

SEQ ID NO: 183 - DNA, 33 bp, Homo sapiens
gacaatgagggtgacayactcactcagttttac

SEQ ID NO: 184 - DNA, 33 bp, Homo sapiens
cctgggggctgagatckaagtgatgaggaggca

SEQ ID NO: 185 - DNA, 33 bp, Homo sapiens
ccatgtgctggcgatayaacagttaacactata

SEQ ID NO: 186 - DNA, 33 bp, Homo sapiens
ccatccgtagcacagayggggcgcaagcgtatt

SEQ ID NO: 187 - DNA, 33 bp, Homo sapiens
gaaatgaaggctgtaarcaagaaaaatgctcat

SEQ ID NO: 188 - DNA, 33 bp, Homo sapiens
tagagcaacaattagayactcctgtctacttcc

SEQ ID NO: 189 - DNA, 33 bp, Homo sapiens
gtgcagttggaagatgyagacgcgcctcgcttg

SEQ ID NO: 190 - DNA, 33 bp, Homo sapiens
gaacaagtgagctcgaygggccagactctaaaa

SEQ ID NO: 191 - DNA, 33 bp, Homo sapiens
gaagaggagagaggcarctttgagaaagccgag

SEQ ID NO: 192 - DNA, 33 bp, Homo sapiens
atttgactgtgtggagractctgtatttgagat

SEQ ID NO: 193 - DNA, 33 bp, Homo sapiens
gagcctttggtaagacwaggttattattccaat

SEQ ID NO: 194 - DNA, 33 bp, Homo sapiens
gatgaacacccatgtgrcacatcaaccaccggc

SEQ ID NO: 195 - DNA, 33 bp, Homo sapiens
gagagggtgagatgcayagaacagtccaggctt

SEQ ID NO: 196 - DNA, 33 bp, Homo sapiens
gagctccaaactgacgkccaggaacatgctctc

SEQ ID NO: 197 - DNA, 33 bp, Homo sapiens
gagggtgaaaggatggkattaaacaaacaaaca

SEQ ID NO: 198 - DNA, 33 bp, Homo sapiens
gaggtgagaatgccaaycatgttgatactaaat

SEQ ID NO: 199 - DNA, 33 bp, Homo sapiens
gctctcaacgcacagakcaattcctccaagagt

SEQ ID NO: 200 - DNA, 33 bp, Homo sapiens
gccctaacttaagatcratttctgattataatc

SEQ ID NO: 201 - DNA, 33 bp, Homo sapiens
gcgcctgtcactccgaygctccactgaagtctc

SEQ ID NO: 202 - DNA, 33 bp, Homo sapiens
gcctcagttttctaaasagtaaggcagtagtca